(19) United States
(12) Patent Application Publication
(10) Pub. No.: US 2009/0286808 A1
(43) Pub. Date: Nov. 19, 2009

(54) Opsin Stabilizing Compounds and Methods of Use

(75) Inventors: Shalesh Kaushal, Gainesville, FL (US); Syed M. Noorwez, Gainesville, FL (US); David A. Ostrov, Gainesville, FL (US)

Correspondence Address: EDWARDS ANGELL PALMER & DODGE LLP, P.O. BOX 55874, BOSTON, MA, 02205, US

(73) Assignee: University of Florida Research Foundation, Inc., Gainesville, FL (US)

(21) Appl. No.: 12/356,967

(22) Filed: Jan. 21, 2009

Related U.S. Application Data
(63) Continuation of application No. PCT/US2007/016989, filed on Jul. 27, 2007.
(60) Provisional application No. 60/833,884, filed on Jul. 27, 2006.
(60) Provisional application No. 60/878,492, filed on Jan. 3, 2007.
(60) Provisional application No. 60/933,345, filed on Jun. 5, 2007.

(51) Int. Cl. A61K 31/4965; A61K 31/505; A61K 31/44; A61K 31/415; A61K 31/11; C07K 1/107

(52) U.S. Cl. 514/255.02; 514/274; 514/352; 514/407; 514/701; 530/402; 530/409

(57) ABSTRACT

The present invention provides compositions and methods useful in the treatment and/or prevention of ophthalmic conditions and diseases, such as retinitis pigmentosa, that are dependent upon or related to misfolded opsin proteins in vivo. In addition, screening assays for agents useful in such treatment methods are described.

### CROSS-REFERENCE TO RELATED APPLICATIONS

This application is related to U.S. Provisional Patent Applications 60/833,884, filed 27 Jul. 2006, 60/878,492, filed 3 Jan. 2007, and 60/933,345, filed Jun. 5, 2007, the disclosures of which are hereby incorporated by reference in their entirety.

### FIELD OF THE INVENTION

The present invention relates to methods of using opsin-binding compounds for the treatment and/or prevention of ophthalmic diseases and conditions and methods of screening for agents useful there for.

### BACKGROUND OF THE INVENTION

The visual cycle (also frequently referred to as the retinoid cycle) comprises a series of light-driven and/or enzyme catalyzed reactions whereby a light-sensitive chromophore (called rhodopsin) is formed by covalent bonding between the protein opsin and the retinoid agent 11-cis-retinal and subsequently, upon exposure to light, the 11-cis-retinal is converted to all-trans-retinal, which can then be regenerated into 11-cis-retinal to again interact with opsin. A number of visual, ophthalmic, problems can arise due to interference with this cycle. It is now understood that at least some of these problems are due to improper protein folding, such as that of the protein opsin.

The main light and dark receptor in the mammalian eye is the rod cell, which contains a folded membrane containing protein molecules that can be sensitive to light, the main one being opsin. Like other proteins present in mammalian cells, opsin is synthesized in the endoplasmic reticulum (i.e., on ribosomes) of the cytoplasm and then conducted to the cell membrane of rod cells. In some cases, such as due to genetic defects and mutation of the opsin protein, opsin can exhibit improper folding to form a conformation that either fails to properly insert into the membrane of the rod cell or else inserts but then fails to properly react with 11-cis-retinal to form native rhodopsin. In either case, the result is moderate to severe interference with visual perception in the animal so afflicted.

Among the diseases and conditions linked to improper opsin folding is retinitis pigmentosa (RP), a progressive ocular-neurodegenerative disease (or group of diseases) that affect an estimated 1 to 2 million people worldwide. In RP, photoreceptor cells in the retina are damaged or destroyed, leading to loss of peripheral vision (i.e., tunnel vision) and subsequent partial or near-total blindness.

In the American population the most common defect occurs as a result of replacement of a proline residue by a histidine residue at amino acid number 23 in the opsin polypeptide chain (dubbed “P23H”), caused by a mutation in the gene for opsin. The result is production of a destabilized form of the protein, which is misfolded and aggregates in the cytoplasm rather than being transported to the cell surface. Like many other protein conformational diseases (PCDs), the clinically common P23H opsin mutant associated with autosomal dominant retinitis pigmentosa is misfolded and retained intracellularly. The aggregation of the misfolded protein is believed to result in photoreceptor damage and cell death.

Recent studies have identified small molecules that stabilize misfolded mutant proteins associated with disease. Some of these, dubbed “chemical chaperones,” stabilize proteins non-specifically. Examples of these include glycerol and trimethylamine oxide. These are not very desirable for treating ophthalmic disease because such treatment usually requires high dosages that may cause toxic side effects. Other agents, dubbed “pharmacological chaperones,” (which include native ligands and substrate analogs) act to stabilize the protein by binding to specific sites and have been identified for many mis-folded proteins, e.g., G-protein coupled receptors. Opsin is an example of a G-protein coupled receptor and its canonical pharmacological chaperones include the class of compounds referred to as retinoids. Thus, certain retinoid compounds have been shown to stabilize mutant opsin proteins (see, for example, U.S. Patent Pub. 2004-0242704, as well as Noorwez et al., J. Biol. Chem., 279(16): 16278-16284 (2004)).

Retinoids effective in correcting opsin mis-folding include 11-cis-retinal (the native ligand) as well as 9-cis-retinal (which binds to opsin to form isorhodopsin) and 11-cis-7-ring-retinal (a chemically constrained retinoid that forms a light-stable rhodopsin pigment). These retinoids form a covalent bond with opsin. When such compounds are administered during mutant opsin expression in COS-7 cells or HEK293 cells, they increase both yield and cell surface localization of the protein.

Despite their therapeutic promise, the efficacy of these retinoids in treating rhodopsin retinitis pigmentosa (“RP”) was inconclusive. Encouraging results were obtained with vitamin A palmitate, which was used to treat RP mice possessing a transgenic mutant T17M (Class II) gene, which expresses a misfolded opsin protein, or P347S (Class I) human opsin gene (the latter designations stand for the mutations contained in the gene present in the respective transgenic mouse). This treatment resulted in a 40-45% decrease in the rate of decline in electroretinogram (ERG) a- and b-wave amplitudes due to retinal degeneration in T17M mice. The decrease in outer nuclear layer (ONL) thickness was 24% in treated animals, consistent with less degeneration. However, no significant changes were observed in P347S mice, suggesting that the treatment is specific for misfolded opsins (Class II). Supplementation with vitamin A palmitate has also been tested in RP patients, but with less encouraging results (Berson et al. (2000)). A modest decrease in retinal degeneration was observed over a period of several years, but long-term benefits were not apparent. Moreover, vitamin A and related compounds are potentially toxic (Teelmann et al. (1989)), and teratogenic (Collins et al. (1999)), prohibiting treatment at higher doses. Because of these concerns, there is a need for novel compounds, or at least compounds not heretofore tested, that, like retinoids, stabilize mutant opsins and thereby retard the development of diseases such as RP.

Computer-assisted molecular docking has lead to the successful discovery of novel ligands for more than 30 targets (Shoichet et al. (2002)). This strategy has been applied primarily to enzymes, such as aldose reductase (Iwata et al. (2001), Bcl-2 (Enyedy et al. (2001), matriptase (Enyedy et al. (2001), adenovirus protease (Pang et al. (2001)), AmpC fl-lactamase, carbonic anhydrase (Gruneberg et al. (2002)), HPRTase (Freymann et al. (2000)), dihydrodipicolinate (Paiva et al. (2001)) and Cdk4 (Honma et al. (2001)). Improvements in docking algorithms and multiprocessor resources have improved the technique of computer-assisted molecular docking such that it can now be applied to more challenging problems. For example, this approach has recently been applied to defining small molecules that target protein-protein interfaces, which are relatively broad and flat compared to easily targeted enzyme active sites.

### BRIEF SUMMARY OF THE INVENTION

In one aspect, the invention generally provides a method of correcting the conformation of a mis-folded opsin protein. The method involves contacting a mis-folded opsin protein with an opsin-binding agent that reversibly binds non-covalently to the mis-folded opsin protein, thereby correcting the conformation of the mis-folded opsin protein.

In another aspect, the invention provides a method of ameliorating an ocular protein conformation disease in a subject. The method involves administering to the subject an effective amount of an opsin-binding agent that reversibly binds non-covalently to the mutant opsin protein, thereby ameliorating the ocular protein conformation disease.

In yet another aspect, the invention provides an opthalmologic composition containing an effective amount of an opsin-binding agent in a pharmaceutically acceptable carrier, where the agent reversibly binds non-covalently to opsin protein to prevent retinoid binding in the retinal binding pocket of the opsin.

In yet another aspect, the invention provides an oral dosage form containing the non-retinoid agent of a previous aspect.

In yet another aspect, the invention provides a method of identifying an opsin-binding agent that corrects the conformation of a mis-folded opsin protein. The method involves contacting a mutant opsin protein with an opsin-binding test compound that binds at, in or near the retinal binding pocket of opsin under conditions that promote the binding of the test compound to the mutant opsin protein; and determining that the mutant opsin protein is in the correct conformation, thereby identifying the test compound as an opsin-binding agent that corrects the conformation of a mis-folded opsin protein. In one embodiment, the test compound reversibly binds non-covalently to the retinal binding pocket of the mutant opsin protein and competes with a retinoid for binding the opsin protein.

In another aspect, the invention provides a method of rescuing photoreceptor function in a mammalian eye containing a mis-folded opsin protein. The method involves contacting the mis-folded opsin protein with an opsin-binding agent that reversibly binds non-covalently to the mis-folded opsin protein, thereby rescuing photoreceptor function in the mammalian eye.

In another aspect, the invention provides a method for treating or preventing an ocular protein conformation disease in a subject, the method involves administering to a subject having or at risk of developing an ocular protein conformation disease a therapeutically effective amount of an opsin-binding agent selected from the group consisting 1-(3,5-dimethyl-1H-pyrazol-4-yl)-ethanone, 1-furan-2-ylmethyl-2,4-dioxo-1,2,3,4-tetrahydro-pyrimidine-5-carbonitrile, phenyl-phosphinic acid, 2-methyl-4-nitro-pyridine, 3,6-bis-(2-hydroxyethy)-piperazine-2,5-dione, diisopropylaminoacetonitrile, 3,4-methylenedioxybenzonitrile, diethyl(2-mercaptoethyl)amine, 6-imino-1-methyl-1,6-dihydro-3-pyridinecarboxamide, 1H-1,2,3-benzotriazol-1-amine, 4-salicylideneamino-1,2,4-triazole, β-ionone, cis-1,3-dimethylcyclohexane, and a pharmaceutically acceptable salt, hydrate, or solvate thereof.

In various embodiments of any of the preceding aspects, the ocular protein conformation disorder is any one or more of wet or dry form of macular degeneration, retinitis pigmentosa, a retinal or macular dystrophy, Stargardt's disease, Sorsby's dystrophy, autosomal dominant drusen, Best's dystrophy, peripherin mutation associate with macular dystrophy, dominant form of Stargart's disease, North Carolina macular dystrophy, light toxicity, and retinitis pigmentosa. In another embodiment of the preceding aspects, the method further involves administering to the patient at least one additional agent selected from the group consisting of a proteasomal inhibitor, an autophagy inhibitor, a lysosomal inhibitor, an inhibitor of protein transport from the ER to the Golgi, an Hsp90 chaperone inhibitor, a heat shock response activator, a glycosidase inhibitor, and a histone deacetylase inhibitor, where the opsin-binding compound and the additional compound are administered simultaneously or within fourteen days of each other in amounts sufficient to treat the subject. In another embodiment of the preceding aspects, the subject contains a mutation that effects protein folding. In another embodiment of the preceding aspects, the mutation is in an opsin (e.g., a P23H mutation). In other embodiments of the preceding aspects, the non-retinoid opsin-binding agent is selected from the group consisting of 1-(3,5-dimethyl-1H-pyrazol-4-yl)-ethanone, 1-furan-2-ylmethyl-2,4-dioxo-1,2,3,4-tetrahydro-pyrimidine-5-carbonitrile, phenyl-phosphinic acid, 2-methyl-4-nitro-pyridine, 3,6-bis-(2-hydroxyethy)-piperazine-2,5-dione, diisopropylaminoacetonitrile, 3,4-methylenedioxybenzonitrile, diethyl(2-mercaptoethyl)amine, 6-imino-1-methyl-1,6-dihydro-3-pyridinecarboxamide, 1H-1,2,3-benzotriazol-1-amine, 4-salicylideneamino-1,2,4-triazole, β-ionone, cis-1,3-dimethylcyclohexane, and a pharmaceutically acceptable salt, solvate, or hydrate thereof. In still other embodiments of the preceding aspects, the opsin-binding compound and the additional compound are administered within one, three, five, ten, or fourteen days of each other. In still other embodiments, the opsin-binding compound and the additional compound are administered simultaneously. In another embodiment of the preceding aspects, the opsin-binding compound and the additional compound are administered directly to the eye. In another embodiment of the preceding aspects, the administration is intra-ocular. In another embodiment the opsin-binding compound and the additional compound are each incorporated into a composition that provides for their long-term release. In another embodiment of the preceding aspects, the composition is part of a microsphere, nanosphere, or nano emulsion. In another embodiment, the composition is administered via a drug-delivery device that effects long-term release. In another embodiment, the method further involves administering a vitamin A supplement.

In another aspect, the invention provides a method of increasing the amount of biochemically functional opsin protein in a photoreceptor cell. The method involves contacting a photoreceptor cell with an effective amount of an opsin-binding agent that reversibly binds non-covalently to an opsin protein in the cell, thereby increasing the level of biochemically functional conformation of opsin protein.

In yet another aspect, the invention provides a method of correcting the conformation of a mis-folded opsin protein. The method involves contacting a mis-folded opsin protein with a retinoid opsin-binding agent that binds to the mis-folded opsin protein in the retinal binding pocket of the opsin, thereby correcting the conformation of the mis-folded opsin protein.

In yet another aspect, the invention provides a method of stabilizing a mutant opsin protein in a wild-type protein conformation, involving contacting the mutant opsin protein with an opsin-binding agent that reversibly binds non-covalently to the mutant opsin protein, thereby stabilizing the mutant opsin protein in a wild-type protein conformation.

In various embodiments of any of the above aspects, the opsin binding agent is selective for opsin. In another embodiment, the opsin-binding agent competes with a retinoid for binding to the opsin. In another embodiment, the opsin-binding agent binds in the retinal binding pocket of the opsin. In other embodiments of the above aspects, the opsin-binding agent binds to the opsin protein so as to inhibit covalent binding of 11-cis-retinal to the opsin protein when the 11-cis-retinal is contacted with the opsin protein when the non-retinoid opsin-binding agent is present. In still other embodiments of the above aspects, the opsin-binding agent is a retinoid or a non-retinoid. In still other embodiments of the above aspects, the opsin-binding agent binds reversibly. In still other embodiments of the above aspects, the opsin-binding agent binds covalently or non-covalently. In one embodiment, the mis-folded opsin protein is present in a cell (e.g., a mammalian eye, such as a human eye). In other embodiments of the above aspects, the contacting occurs while the mis-folded opsin is present in the endoplasmic reticulum of the cell. In other embodiments of the above aspects, the mis-folded opsin protein contains a mutation in its amino acid sequence, such as any one or more of T17M, P347S and P23H. Preferably, the mutation is P23H. In other embodiments of the above aspects, the opsin-binding agent is any one or more of 1-(3,5-dimethyl-1H-pyrazol-4-yl)-ethanone, 1-furan-2-ylmethyl-2,4-dioxo-1,2,3,4-tetrahydro-pyrimidine-5-carbonitrile, phenyl-phosphinic acid, 2-methyl-4-nitro-pyridine, 3,6-bis-(2-hydroxyethy)-piperazine-2,5-dione, diisopropylaminoacetonitrile, 3,4-methylenedioxybenzonitrile, diethyl(2-mercaptoethyl)amine, 6-imino-1-methyl-1,6-dihydro-3-pyridinecarboxamide, 1H-1,2,3-benzotriazol-1-amine, 4-salicylideneamino-1,2,4-triazole, β-ionone, cis-1,3-dimethylcyclohexane, and a pharmaceutically acceptable salt, hydrate, or solvate thereof. In still other embodiments, the opsin-binding agent is selective for binding to opsin In still other embodiments, the opsin-binding agent competes with a retinoid for binding to the retinal binding pocket. In still other embodiments, the opsin-binding agent binds in the retinal binding pocket of the opsin. In still other embodiments, the opsin-binding agent binds to the opsin protein so as to inhibit covalent binding of 11-cis-retinal to the opsin protein when the 11-cis-retinal is contacted with the opsin protein when the non-retinoid opsin-binding agent is present. In still other embodiments of the above aspects, opsin-binding agent is a non-retinoid. In still other embodiments of the above aspects, contacting occurs by administering the opsin-binding agent to a mammal identified as having reduced photoreceptor function. In still other embodiments of the above aspects, the administering is by topical administration, by local (e.g., intraocular injection or periocular injection), or by systemic administration (e.g., oral). In still other embodiments of the above aspects, a mis-folded opsin protein contains a mutation in its amino acid sequence, such as T17M, P347S and P23H. In still other embodiments of any of the above aspects, the subject (e.g., a mammal) has or has a propensity to develop an ocular protein conformation disease that is any one or more of a wet or dry form of age-related macular degeneration, retinitis pigmentosa, retinal or macular dystrophy, Stargardt's disease, Sorsby's dystrophy, autosomal dominant drusen, Best's dystrophy, peripherin mutation associated with macular dystrophy, a dominant form of Stargart's disease, North Carolina macular dystrophy, light toxicity, and retinitis pigmentosa. In still other embodiments of any of the above aspects, the contacting occurs in a eukaryotic cell (e.g., a mammalian cell or human cell) expressing the mutant opsin protein. In other embodiments, the cell is a recombinant cell engineered to express the mutant opsin protein. In other embodiments of the above aspects, the correct conformation of the opsin is determined by assaying absorbance at 500 nm, by assaying visual function using an electroretinogram, in a histological assay, by monitoring opsin protein localization, or by assaying retinal morphology. In still other embodiments, an opsin-binding agent binds in the retinal binding pocket of the opsin. In still other embodiments of the above aspects, the contacting occurs in vitro or in vivo. In still other embodiments of the above aspects, the cell (e.g., human cell) is a rod or cone cell.

Because formation of the native opsin conformation facilitates binding of 11-cis-retinal to said opsin to form the visual chromophore, determination that the mutant or mis-folded opsin is in the native conformation is readily achieved by any method that reveals such reaction. One non-limiting example is contacting the opsin of step (b) above with 11-cis-retinal and measuring formation of the chromophore of the resulting rhodopsin at 500 nm. Formation of rhodopsin could also be determined using antibodies specific for native rhodopsin.

In such methods, the test compound reversibly binds non-covalently to the retinal binding pocket of said mutant opsin protein to prevent retinoid binding to said mutant opsin protein and is selective for binding to opsin.

The present invention also offers a method for treating or preventing retinitis pigmentosa in a patient, comprising administering to a patient afflicted with, or at risk of developing, retinitis pigmentosa a therapeutically effective amount of a non-retinoid opsin-binding agent that shows positive activity in the screening methods of the invention. Such methods may further comprise administering to said patient at least one additional agent selected from the group consisting of a proteasomal inhibitor, an autophagy inhibitor, a lysosomal inhibitor, an inhibitor of protein transport from the ER to the Golgi, an Hsp90 chaperone inhibitor, a heat shock response activator, a glycosidase inhibitor, and a histone deacetylase inhibitor, wherein the opsin-binding compound and the additional compound are administered simultaneously or within fourteen days of each other in amounts sufficient to treat the subject. Preferably, such mutation affects protein folding.

The present invention also provides a method of increasing the amount of biochemically functional opsin in a cell, comprising:

a) contacting a cell with an effective amount of a non-retinoid opsin-binding agent having positive activity in the method of claim 55, and

b) identifying an increase in the amount of a biochemically functional conformation of the protein. In a specific embodiment of this method, the cell is further contacted with at least one compound selected from the group consisting of: a proteasomal inhibitor, an autophagy inhibitor, a lysosomal inhibitor, an inhibitor of protein transport from the ER to the Golgi, an Hsp90 chaperone inhibitor, a heat shock response activator, a glycosidase inhibitor, and a histone deacetylase inhibitor. Preferably, the cell is a mammalian and/or recombinant cell and comprises a mutant opsin protein.

### BRIEF DESCRIPTION OF THE DRAWINGS

FIGS. 1A-1F show that β-ionone inhibited opsin regeneration and rescued mutant opsin. (a) Purified wild-type (WT) opsin when regenerated with 11-cis-retinal formed a 500 nm absorbing pigment. Formation of this pigment was inhibited by β-ionone. (b) β-ionone did not form a 500 nm absorbing pigment with opsin. (c) β-ionone stabilized misfolded opsin. Very little pigment was obtained from cells carrying the P23H mutation. No pigment was generated under control conditions [symbol] but the presence of β-ionone increased the yield of P23H pigment by 2.5-fold [symbol] About a 5-fold increase in pigment was obtained in the presence of 11-cis-retinal [symbol] (d) β-ionone increased the yield of total P23H opsin, but more protein was properly folded (purified opsin). (e) No pigment was generated [symbol] on providing β-ionone to HEK cells expressing the mutant P23H opsin, but regeneration of opsin by incubating β-ionone treated cells with 11-cis-retinal [symbol] lead to formation of pigment. (f) Presence of cis-1,3-dimethylcyclohexane, another inhibitor of opsin regeneration, also resulted in increased yield of folded P23H rhodopsin [symbol] (control shown as unbroken line).

FIGS. 2A, 2B, 2C, and 2D show that compound SN10011 inhibited opsin regeneration and rescued mutant opsin. (a) Pigment formation of WT opsin with 11-cis-retinal was inhibited by SN10011 at 2 mM and 5 mM concentrations. (b) No 500 nm absorbing pigment was generated by SN10011 with 11-cis-retinal in vitro and (c) the compound did not absorb in the visible spectrum. (d) SN10011 led to increased yield of folded rhodopsin [symbol] over that in the absence of the compound

[symbol]

FIGS. 3A-3C show the molecular docking strategy for the compounds of the invention. FIG. 3 A shows the retinal binding pocket of human opsin. FIG. 3B shows binding of β-ionone in the pocket FIG. 3C shows binding of compound SN10011 in the retinal pocket.

### DEFINITIONS

Unless expressly stated otherwise elsewhere herein, the following terms have the stated meaning with respect to the present invention.

By “proteasomal inhibitor” is meant a compound that reduces a proteasomal activity, such as the degradation of a ubiquinated protein.

By “autophagy inhibitor” is meant a compound that reduces the degradation of a cellular component by a cell in which the component is located.

By “lysosomal inhibitor” is meant a compound that reduces the intracellular digestion of macromolecules by a lysosome. In one embodiment, a lysosomal inhibitor decreases the proteolytic activity of a lysosome.

By “Inhibitor of ER-Golgi protein transport” is meant a compound that reduces the transport of a protein from the ER (endoplasmic reticulum) to the Golgi, or from the Golgi to the ER.

By “HSP90 chaperone inhibitor” is meant a compound that reduces the chaperone activity of HSP90. In one embodiment, the inhibitor alters protein binding to an HSP90 ATP/ADP pocket.

By “heat shock response activator” is meant a compound that increases the chaperone S activity or expression of a heat shock pathway component Heat shock pathway components include, but are not limited to, HSP100, HSP90, HSP70, HASP60, HSP40 and small HSP family members.

By “glycosidase inhibitor” is meant a compound that reduces the activity of an enzyme that cleaves a glycosidic bond.

By “histone deacetylase inhibitor” is meant a compound that reduces the activity of an enzyme that deacetylates a histone.

By “reduces” or “increases” is meant a negative or positive alteration, respectively, of at least 10%, 25%, 50%, 75%, or 100%.

As used herein, the phrase “biochemically functional conformation” means that a protein has a tertiary structure that permits the protein to be biologically active. When a mutant protein assumes a biochemically functional conformation its biological activity is increased. Accordingly, a mutant protein having a biochemically functional conformation may, to some degree, functionally substitute for a wild-type protein.

As stated herein, the term “wild-type conformation” refers to the 3 dimensional conformation or shape of a protein that is free of mutations present in its amino acid sequence that affect the conformation or shape of the protein, such that protein function is altered relative to wild-type protein function. For opsin, a wild-type conformation is a conformation that is free from mutations that cause mis-folding, such as the mutation designated P23H (P23H opsin) (see, for example, GenBank Accession Nos. NM_{—}000539 and NP_{—}000530) (meaning that a proline is replaced by a histidine at residue 23 starting from the N-terminus). Opsin in a “wild-type conformation” is capable of opsin biological function, including but not limited to, retinoid binding, visual cycle function, and insertion into a photoreceptor membrane.

By “correcting the conformation” of a protein is meant inducing the protein to assume a conformation having at least one biological activity associated with a wild-type protein.

By “mis-folded opsin protein” is meant a protein whose tertiary structure differs from the conformation of a wild-type protein, such that the misfolded protein lacks one or more biological activities associated with the wild-type protein.

By “opsin-binding agent” is meant a small molecule, polypeptide, or polynucleotide, or fragment thereof, capable of binding to an opsin polypeptide. In one embodiment, the agent is a retinoid that binds opsin non-covalently and reversibly. In another embodiment, the agent is a non-retinoid small compound that binds reversibly to opsin. The term “retinoid” refers to a diterpene having a non-aromatic 6-member ring core hydrocarbon structure and an eleven carbon side chain. Exemplary retinoids include 11-cis-retinal and all-trans-retinal.

By “selectively binds” is meant a compound that recognizes and binds a polypeptide of the invention, but which does not substantially recognize and bind other molecules in a sample, for example, a biological sample.

By “competes for binding” is meant that a compound of the invention and an endogenous ligand bind to the same site on a target molecule and are, therefore, incapable of occupying that site at the same time. Assays to measure competitive binding are known in the art, and include, measuring a dose dependent inhibition in binding of a compound of the invention and an endogenous ligand by measuring t_{1/2}, for example.

As used herein, the term “pharmaceutically acceptable salt,’ is a salt formed from an acid and a basic group of one of the compounds of the invention (e.g., of Table 1 or 2, or beta-ionone or Cis-1,3-dimethylcyclohexane). Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts.

The term “pharmaceutically acceptable salt” also refers to a salt prepared from a compound of the invention (e.g., of Table 1 or Table 2, or β-ionone, SN10011, or Cis-1,3-dimethylcyclohexane) having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl-N-ethylamine; diethylamine; triethylamine; mono-, bis-, or tris-(2-hydroxy-lower alkylamines), such as mono-, bis-, or tris-(2-hydroxyethyl)-amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)-amine, or tri-(2-hydroxyethyl)amine; N-methyl-D-glucamine; and amino acids such as arginine, lysine, and the like.

The term “pharmaceutically acceptable salt” also refers to a salt prepared from a compound disclosed herein, e.g., a compound of Table 1 or Table 2 or β-ionone, SN10011, or Cis-1,3-dimethylcyclohexane, having a basic functional group, such as an amino functional group, and a pharmaceutically acceptable inorganic or organic acid. Suitable acids include, but are not limited to, hydrogen sulfate, citric acid, acetic acid, oxalic acid, hydrochloric acid, hydrogen bromide, hydrogen iodide, nitric acid, phosphoric acid, isonicotinic acid, lactic acid, salicylic acid, tartaric acid, ascorbic acid, succinic acid, maleic acid, besylic acid, fumaric acid, gluconic acid, glucaronic acid, saccharic acid, formic acid, benzoic acid, glutamic acid, methanesulfonic acid, ethanesulfonic acid, benzenesulfonic acid, and p-toluenesulfonic acid.

The term “pharmaceutically-acceptable excipient” as used herein means one or more compatible solid or liquid tiller, diluents or encapsulating substances that are suitable for administration into a human.

The term “carrier” denotes an organic or inorganic ingredient, natural or synthetic, with which the active ingredient is combined to facilitate administration.

The term “parenteral” includes subcutaneous, intrathecal, intravenous, intramuscular, intraperitoncal, or infusion.

### DETAILED DESCRIPTION OF THE INVENTION

In accordance with the present invention, it has been found that certain compounds are capable of binding to and stabilizing a conformation of a mutant P23H rhodopsin which permits the mutant protein to form a stable complex with 11-cis-retinal (such as a biochemically functional conformation). Such compounds are referred to herein as “opsin-binding” or “opsin-stabilizing” compounds, or alternatively as pharmacological “chaperones” which can stabilize opsins.

Certain synthetic retinoids (compounds structurally related to retinol (Vitamin A alcohol)) have been reported to bind to opsin. In the embodiments of the present invention, opsin-binding compounds are not synthetic or naturally-occurring retinoids (that is, the opsin-binding compounds are not structurally analogous to retinol or retinal, e.g., the opsin binding compounds of the invention may lack a polyene chain and/or may lack a trimethylcyclohexene moiety). For purposes of this invention, beta-ionone is considered a non-retinoid and, in certain embodiments, is contemplated for use in the inventive methods and compositions. In certain embodiments, an opsin-binding compound is a non-polymeric (e.g., a small molecule) compound having a molecular weight less than about 1000 daltons, less than 800, less than 600, less than 500, less than 400, or less than about 300 daltons. In certain embodiments, an opsin-binding compound can increase the yield (e.g., from or in a cell) of a stably-folded and/or complexed mutant protein by at least 10%, 15%, 20%, 25%, 50%, 75%, or 100% compared to an untreated control cell or protein.

Examples of opsin-binding or opsin-stabilizing compounds include 1-(3,5-dimethyl-1H-pyrazol-4-yl)-ethanone, 1-furan-2-ylmethyl-2,4-dioxo-1,2,3,4-tetrahydro-pyrimidine-5-carbonitrile, phenyl-phosphinic acid, 2-methyl-4-nitro-pyridine, 3,6-bis-(2-hydroxyethy)-piperazine-2,5-dione, diisopropylaminoacetonitrile, 3,4-methylenedioxybenzonitrile, diethyl(2-mercaptoethyl)amine, 6-imino-1-methyl-1,6-dihydro-3-pyridinecarboxamide, 1H-1,2,3-benzotriazol-1-amine, 4-salicylideneamino-1,2,4-triazole, β-ionone, Cis-1,3-dimethylcyclohexane, and a pharmaceutically acceptable salt thereof.

The invention features compositions and methods that are useful for correcting misfolded proteins (e.g., opsin proteins) in vivo.

The invention is generally based on the discovery that certain non-retinoid compounds can be used to correct the conformation of a misfolded protein (e.g., misfolded opsin protein) or to increase the amount of correctly folded protein in a cell. Without wishing to be bound by any particular theory, these compounds are believed to stabilize mutant opsin by binding to the opsin, e.g., at, in or near the retinal binding site.

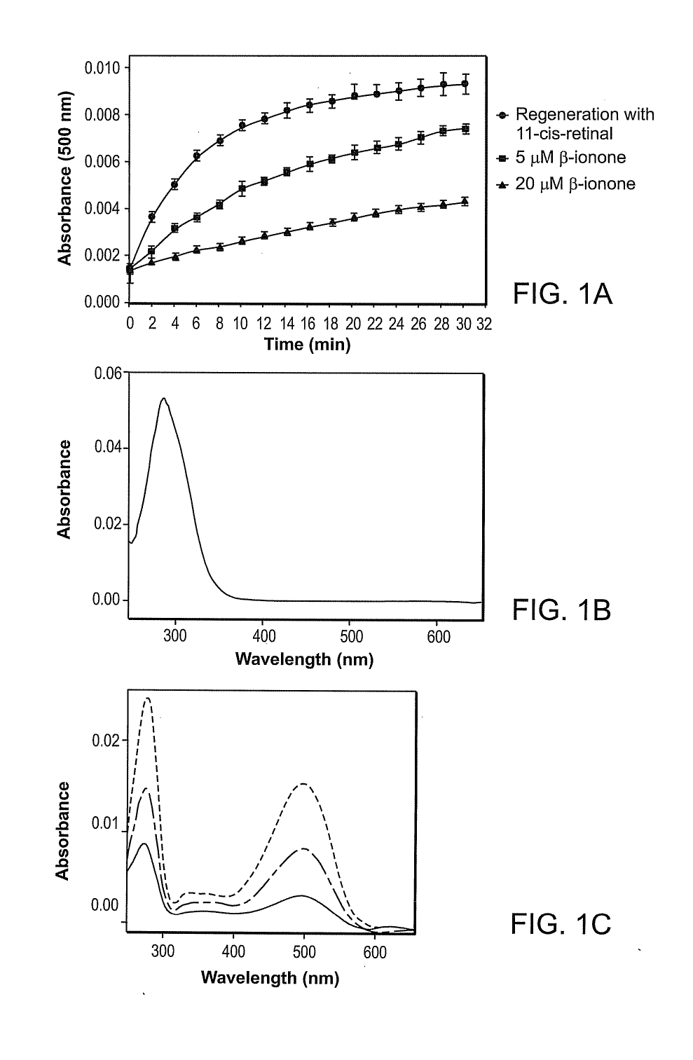

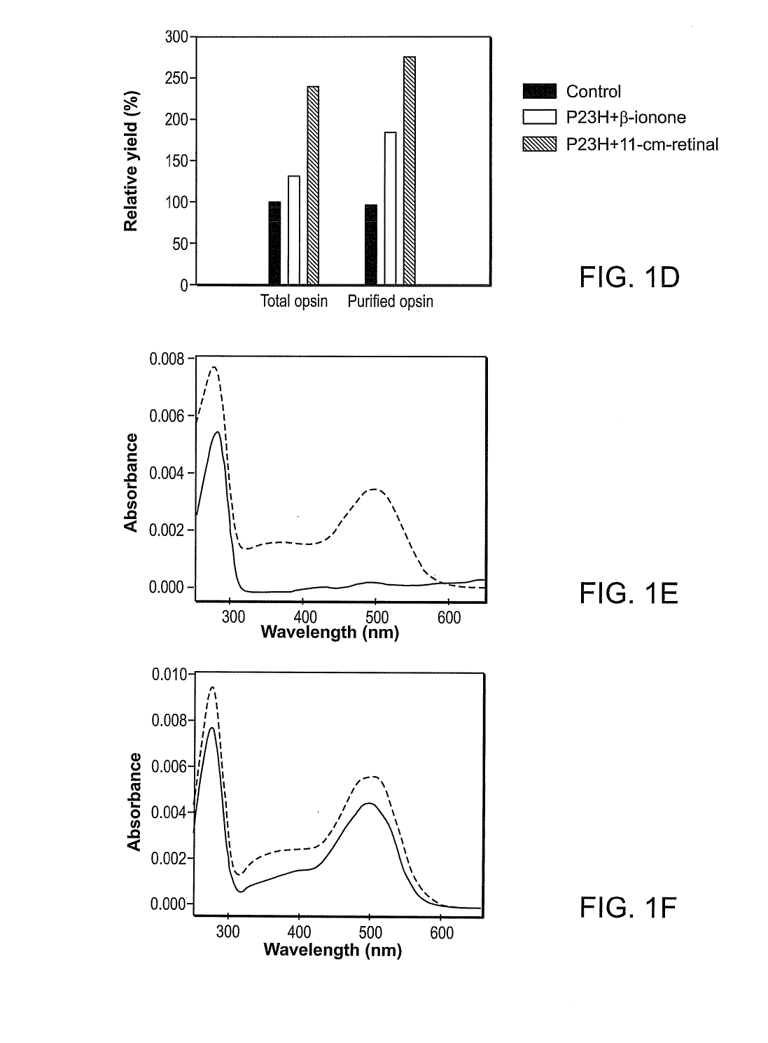

Opsin, the GPCR (G-protein coupled receptor) responsible for vision, readily regenerates with 11-cis-retinal to form the visual pigment rhodopsin. The pigment is generated by formation of a protonated Schiff base between the aldehyde group of 11-cis-retinal and the ε-amino group of L-lysine in opsin (Matsumoto and Yoshizawa, Nature. 1975 Dec. 11; 258(5535):523-6). β-ionone (structure in Example 4) carries the six-membered ring configuration of retinal but has a shorter side chain (Daemen, Nature 1978 Dec. 21-28; 276(5690):847-8) and hence effectively competes with 11-cis-retinal for the chromophore binding site (Matsumoto & Yoshizawa, supra; Daemen supra, Kefalov J Gen Physiol. 1999 March; 113(3):491-503). Under the experimental conditions described below, β-ionone inhibited opsin regeneration in a dose dependent manner demonstrating that β-ionone competitively inhibits retinal binding to opsin (FIG. 1a). The t_{1/2} of pigment formation was determined in the presence and absence of β-ionone (see Example 4). In the absence of β-ionone, pigment formation occurred with a t_{1/2} of 5 minutes. The presence of β-ionone increased the t_{1/2} to 10 minutes in the presence of 5 μM β-ionone and 16 minutes in the presence of 20 μM β3-ionone, respectively. The increase in t_{1/2} was taken as evidence that β-ionone competed with 11-cis-retinal for the retinal binding site of opsin. Further, we determined that no 500 nm absorbing pigment was formed upon addition of β-ionone to purified wild-type opsin (FIG. 1b).

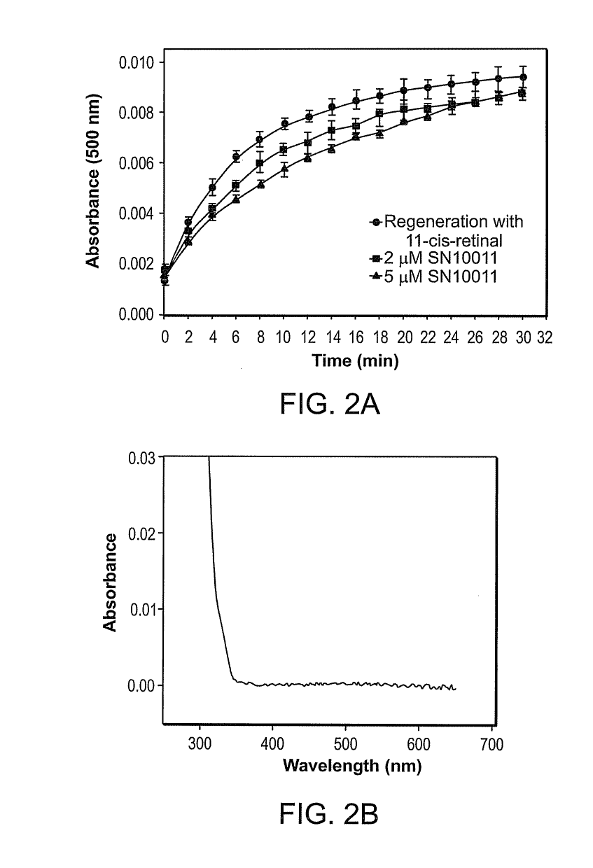

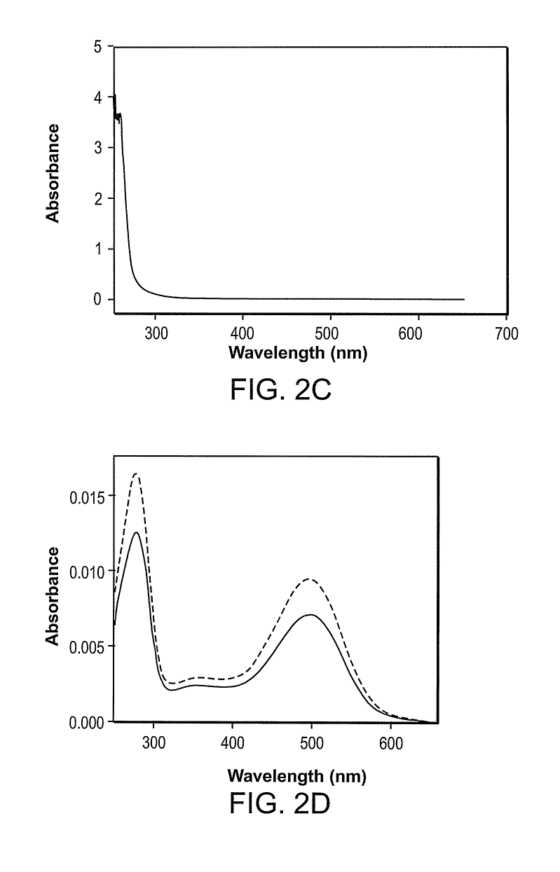

In accordance with the ability of β-ionone to occupy the retinal binding pocket of opsin in vitro and experiments using retinoids to assist and stabilize P23H opsin, β-ionone serves as a pharmacological chaperone. This was shown by adding β-ionone at the time of induction to cells expressing P23H mutant opsin and incubating the cells for 48 hours. Rhodopsin was then purified under conditions that selectively yield properly folded, 11-cis-retinal bound opsin. Results showed that treatment with β-ionone led to a 2.5-fold increase in pigment (long dash) over the control levels (solid line) as shown in FIG. 2C. The presence of 11-cis-retinal led to a 5-fold increase in pigment yield in a similar experiment (short dash) (FIG. 2C). The total yield of opsin in the presence or absence of β-ionone and 11-cis-retinal showed that β-ionone increased the yield of total opsin by 30% relative to the yield of total opsin obtained without addition of any compound (FIG. 2d). Although there was only an insignificant difference in the levels of total opsin and purified properly folded opsin in the case of 11-cis-retinal treatment, there was a pronounced difference when β-ionone was present. The higher increase in properly folded opsin levels indicated that β-ionone stabilizes the opsin molecule in the correct conformation. Thus, β-ionone increased the total amount of opsin within the cell, and also caused a striking increase in the amount of correctly folded P23H rhodopsin. One method of determining total opsin in a cell is described in Example 3. The data reported herein also showed that 11-cis-retinal was about 2-fold more effective than β-ionone in increasing the cellular yields of P23H rhodopsin (FIGS. 1C and 1D). Without wishing to be bound by theory, this difference probably reflects the inability of β-ionone to form stable Schiff base linkage with lysine 296 in the protein.

In accordance with the invention, because HEK293 cells are known to possess a retinoid processing machinery, opsin was purified from β-ionone treated cells and spectroscopically analyzed for formation of pigment to determine whether β-ionone is processed by the cells to form any pigment. No pigment was observed when opsin was purified from β-ionone treated cells (solid line in FIG. 1D). Pigment was observed only after treating the cells with 11-cis-retinal (dashed line) (FIG. 1E). To further test the hypothesis that compounds that non-covalently bind to the chromophore binding site lead to pharmacological rescue of the mutant protein, rhodopsin was purified from P23H opsin expressing cells that were treated with Cis-1,3-dimethylcyclohexane, a much weaker inhibitor of opsin regeneration than 11-cis-retinal. Cis-1,3-dimethylcyclohexane led to a 15% increase in the yield of P23H rhodopsin (FIG. 1F). The lower yield of rhodopsin in the presence of this compound reflects its weaker inhibitory capacity.

Thus, the present invention provides methods of discovery and use of small compounds that fit into the retinal binding pocket of opsin and compete with 11-cis-retinal in vitro, such compounds are, therefore, good pharmacological chaperones.

Molecular docking studies were used to identify candidate compounds that stabilize the retinal binding pocket of rhodopsin and that could be used for further study of the chemical and physical characteristics of such molecules for development of high throughput screening methods for compounds having therapeutic activity.

In accordance with the present invention, β-ionone interacts directly with the retinal binding pocket, so using computer assisted molecular docking, we docked β-ionone into the retinal binding pocket to determine the degree of structural complementarity necessary for enhancing opsin folding. We utilized the crystal structure of rhodopsin to provide the basis for molecular docking and selected the site for molecular docking based on the position of retinal bound to rhodopsin. We then calculated a scoring grid base to encompass the region around the selected site for molecular docking, and subsequently used DOCK 5.1 (UCSF) to position β-ionone. The orientation of β-ionone posed by DOCK 5.1 showed that polar interactions and van der Waals contacts were involved in the specific interactions with opsin.

To identify non-retinoid compounds that could be useful therapeutic agents, we performed molecular docking using a large chemical library of drug-like small molecules in the National Cancer Institute Developmental Therapeutics Program. DOCK5.1 (UCSF) was used to position each one of 20,000 drug-like compounds into the selected site. Each compound was positioned in 100 different orientations, and the best scoring orientations were obtained. Unlike previous molecular docking strategies, each docked compound was selected based on chemical criteria: the Lipinski rules for drug likeness. Therefore, this strategy eliminates compounds that are less likely to be developed into therapeutic agents. The fifth highest scoring compound was 1-(3,5-dimethyl-1H-pyrazol-4-yl)ethanone (Compound 1), SN10011, when in the orientation posed by DOCK5.1 (UCSF) at or in the retinal binding pocket based on the crystal structure of rhodopsin.

Methods of the Invention

The present invention provides a method of restoring the native conformation of a mis-folded opsin protein, comprising contacting said mis-folded opsin protein with a non-retinoid opsin-binding agent that reversibly binds non-covalently (for example, at or in the retinal binding pocket) to said mis-folded opsin protein to prevent retinoid binding in said binding pocket, thereby restoring the native conformation of said mis-folded opsin protein.

The methods may be carried out in vitro or in vivo and the opsin protein may be in a medium, such as a buffer, or may be contained within a cell. Such cell is commonly a mammalian cell, such as a human cell, and may also be a recombinant cell or part of a cell line having selected biochemical or physiological properties. In one preferred embodiment, the cell is an ocular cell, such as a retinal cell. Preferably, the cell is a vertebrate or mammalian (e.g., a human) photoreceptor cell (e.g., a rod cell, a cone cell). In one embodiment, the rod cell is present in a mammalian eye, such as a human eye.

In mammalian cells, proteins are commonly synthesized in the endoplasmic reticulum (ER) of the cell on the ribosomes (i.e., the rough ER or RER). Where a protein is mis-folded, such as due to the presence of a mutation in the gene of the protein that has been translated into a mutated amino acid sequence, such as where a point mutation has occurred and a single amino acid difference is present, said mutation is present when the protein is initially synthesized in the endoplasmic reticulum (ER). Thus, the contacting of the protein, such as opsin, with an agent of the invention may, if the contacting occurs inside a cell, occur in the ER of the cell or where the opsin protein is still attached to a ribosome (and is thus a nascent protein at the time of binding to a compound disclosed herein). Mis-folding of the opsin protein has the consequence of reducing the ability of the opsin to bind 11-cis-retinal to form light sensitive rhodopsin, as well as disrupting the ability of the opsin to correctly insert into the membrane of the rod cell producing it. In addition, an agent of the invention can contact opsin elsewhere in the cell. In one embodiment of the methods of the invention, where the opsin is present in a cell, the contacting occurs while said mis-folded opsin is present in the endoplasmic reticulum of said cell.

In specific embodiments of the methods of the invention, the mis-folded opsin protein comprises a mutation in its amino acid sequence, for example, one of the mutations T17M, P347S or P23H, preferably P23H.

Preferably, in any of the methods of the invention, the opsin-binding agent binds to said opsin in said retinal binding pocket.

In embodiments of any of the compositions and methods of the invention, the opsin-binding agent (e.g., a non-retinoid binding agent) is selective for binding to opsin. Such selectivity is not to be taken as requiring exclusivity that said agent may bind to other proteins as well as to opsin but its binding to opsin will be at least selective, whereby the binding constant (or dissociation constant) for binding to opsin will be lower than the average value for binding to other proteins that also bind retinoids, such as retinal analogs. Preferably, opsin binding agents are non-retinoid opsin-binding agents that bind non-covalently to opsin. Preferably, the opsin binding agent binds at or near the opsin retinal binding pocket, where the native ligand, 11-cis-retinal, normally binds. Without wishing to be bound by theory, in one embodiment the binding pocket accommodates retinal or an agent of the invention, but not both. Accordingly, when an agent of the invention is bound at or near the retinal binding pocket, other retinoids, such as 11-cis-retinal, are unable to bind to opsin. Binding of an agent of the invention inside the retinal binding pocket of a mis-folded opsin molecule serves to direct formation of the native or wild-type conformation of the opsin molecule or to stabilize a correctly folded opsin protein, thereby facilitating insertion of the now correctly-folded opsin into the membrane of a rod cell. Again, without wishing to be bound by theory, said insertion may help to maintain the wild-type conformation of opsin and the opsin-binding agent is free to diffuse out of the binding pocket, whereupon the pocket is available for binding to retinal to form light-sensitive rhodopsin.

Other methods of the invention provide a means to restore photoreceptor function in a mammalian eye containing a mis-folded opsin protein that causes reduced photoreceptor function, comprising contacting said mis-folded opsin protein with an opsin-binding agent (e.g., a non-retinoid) that reversibly binds (e.g., that binds non-covalently) at or near the retinal binding pocket. In other embodiments, binding of the opsin-binding agent to the mis-folded opsin protein competes with 11-cis-retinal for binding in said binding pocket. Desirably, binding of the opsin-binding agent restores the native conformation of said mis-folded opsin protein.

In preferred embodiments, the mammalian eye is a human eye. In additional embodiments, said contacting occurs by administering said opsin-binding agent (e.g., non-retinoid) to a mammal afflicted with an ophthalmic condition, such as a condition characterized by reduced photoreceptor function. In various embodiments, the condition is the wet or dry form of macular degeneration, diabetic retinopathy, a retinal or macular dystrophy, Stargardt's disease, Sorsby's dystrophy, autosomal dominant drusen, Best's dystrophy, peripherin mutation associate with macular dystrophy, dominant form of Stargart's disease, North Carolina macular dystrophy, light toxicity (e.g., due to retinal surgery), or retinitis pigmentosa. The administration may be topical administration or by systemic administration, the latter including oral administration, intraocular injection or periocular injection. Topical administration can include, for example, eye drops containing an effective amount of an agent of the invention in a suitable pharmaceutical carrier.

In another embodiment, the present invention also provides a method of stabilizing a mutant opsin protein, comprising contacting said mutant opsin protein with a non-retinoid opsin-binding agent that reversibly binds non-covalently (for example, at or in the retinal binding pocket) to said mutant opsin protein to prevent retinoid binding in said binding pocket, thereby stabilizing said mutant opsin protein.

The present invention also provides a method of ameliorating loss of photoreceptor function in a mammalian eye, comprising administering an effective amount of an opsin-binding agent, such as a non-retinoid, to a mammal afflicted with a mutant opsin protein that has reduced affinity for 11-cis-retinal, whereby the opsin binding agent reversibly binds (e.g., non-covalently) to the retinal binding pocket of said mutant opsin, thereby ameliorating loss of photoreceptor function in said mammalian eye. In one embodiment, the contacting occurs by administering said opsin-binding agent to a mammal afflicted with said reduced photoreceptor function, wherein said administering may be by topical administration or by systemic administration, the latter including oral, intraocular injection or periocular injection, and the former including the use of eye drops containing an agent of the invention. Such loss of photoreceptor function may be a partial loss or a complete loss, and where a partial loss it may be to any degree between 1% loss and 99% loss. In addition, such loss may be due to the presence of a mutation that causes mis-folding of the opsin, such as where the mutation is the P23H mutation. In another embodiment, the opsin binding agent is administered to ameliorate an opthalmic condition related to the mislocalization of an opsin protein. In one embodiment, the invention provides for the treatment of a subject having the dry form of age-related macular degeneration, where at least a portion of the opsin present in an ocular photoreceptor cell (e.g., a rod or cone cell) is mislocalized. The mislocalized protein fails to be inserted into the membrane of a photoreceptor cell, where its function is required for vision. Administration of the opsin binding agent to a subject having a mislocalized opsin protein rescues, at least in part, opsin localization. Accordingly, the invention is useful to prevent or treat an ophthalmic condition related to opsin mislocalization or to ameliorate a symptom thereof.

The present invention also provides screening assays for compounds effective in the methods of the invention. In one such embodiment, the invention provides a method of identifying an opsin-binding agent that stabilizes a mutant opsin protein in the native conformation of wild-type opsin or increases the amount of correctly folded opsin or rhodopsin in an ocular cell. The method involves:

(a) contacting a mutant opsin protein with an opsin-binding test compound (e.g., a non-retinoid, or a retinoid that fails to form or is incapable of forming a covalent bond with opsin) under conditions that promote the binding of the test compound to the mutant opsin protein (e.g., at or near the retinal binding pocket of opsin); and

(b) determining that said mutant opsin protein is in the native conformation for wild-type opsin as a result of said contacting,

thereby identifying said test compound as an opsin-binding agent that stabilizes a mutant opsin protein in the native conformation of non-mutant opsin or increases the amount of correctly folded opsin or rhodopsin in an ocular cell.

The contacting in such a screening assay may be in vitro or in vivo and, in either case, may occur in a cell, such as a eukaryotic cell, expressing said mutant opsin protein. The cell may be a mammalian cell, such as a human cell, and may also be a recombinant cell engineered to express a mutant opsin protein. Preferably, the test compound being screened reversibly binds to the retinal binding pocket of opsin. In one embodiment, the compound is a retinoid that binds non-covalently. In another embodiment, the compound is a retinoid or non-retinoid that competes with 11-cis-retinal for binding to said mutant opsin protein at the retinal binding pocket.

In other embodiments, a candidate compound is identified as useful in the methods of the invention by a screening assay that (i) identifies an increase in the level of correctly folded protein present in a contacted cell relative to the amount present in an untreated control cell; (ii) that increases the total yield of opsin present in a contacted cell relative to the amount present in an untreated control cell; (iii) that increases the level of correctly folded mutant protein by assaying protein absorbance at 500 nm; that increases visual function in a transgenic animal expressing a mutant opsin (e.g., using an electroretinogram (ERG)) relative to the visual function in an untreated control animal; (iv) that reduces opsin mislocalization or increases correctly localized opsin (i.e., opsin that is localized to a photoreceptor membrane) relative to the localization of opsin in an untreated control cell; or (v) that improves retinal morphology or retinal preservation in a histological assay.

The present invention provides a method for treating or preventing an ophthalmic condition or a symptom thereof, including but not limited to, wet or dry form of macular degeneration, retinitis pigmentosa, a retinal or macular dystrophy, Stargardt's disease, Sorsby's dystrophy, autosomal dominant drusen, Best's dystrophy, peripherin mutation associate with macular dystrophy, dominant form of Stargart's disease, North Carolina macular dystrophy, light toxicity (e.g., due to retinal surgery), or retinitis pigmentosa in a subject, such as a human patient, comprising administering to a subject afflicted with, or at risk of developing, one of the aforementioned conditions or another ophthalmic condition related to the expression of a misfolded or mislocalized opsin protein using a therapeutically effective amount of an opsin-binding agent, e.g., an agent that shows positive activity when tested in any one or more of the screening assays of the invention.

Such a method may also comprise administering to said subject at least one additional agent selected from the group consisting of a proteasomal inhibitor, an autophagy inhibitor, a lysosomal inhibitor, an inhibitor of protein transport from the ER to the Golgi, an Hsp90 chaperone inhibitor, a heat shock response activator, a glycosidase inhibitor, and a histone deacetylase inhibitor, wherein the opsin-binding compound and the additional compound are administered simultaneously or within fourteen days of each other in amounts sufficient to treat the subject.

Here again the patient may comprise a mutation that affects protein folding where said mutation(s) causes mis-folding, e.g., in an opsin protein, and may be any of the mutations recited elsewhere herein, such as a P23H mutation. In other embodiments, the patient has an ophthalmic condition that is related to the mislocalization of an opsin protein. The mislocalized opsin fails to insert into the membrane of a photoreceptor cell (e.g., a rod or cone cell). In general, this failure in localization would effect only a portion of the opsin present in an ocular cell of a patient.

In particular examples of the methods of the invention, the opsin-binding compound and the additional compound are administered within ten days of each other, more preferably within five days of each other, even more preferably within twenty-four hours of each other and most preferably are administered simultaneously. In one example, the opsin-binding compound and the additional compound are administered directly to the eye. Such administration may be intra-ocular. In other examples, the opsin-binding compound and the additional compound are each incorporated into a composition that provides for their long-term release, such as where the composition is part of a microsphere, nanosphere, or nano emulsion. In one example, the composition is administered via a drug-delivery device that effects long-term release. Such methods also contemplate administering a vitamin A supplement along with an agent of the invention.

The present invention also encompasses a method of increasing the amount of biochemically functional opsin in a cell, comprising:

a) contacting a cell with an effective amount of an opsin-binding agent (e.g., a non-retinoid agent or a retinoid that fail to form a covalent bond with opsin) having positive activity in one or more of the screening assays of the invention, and

b) identifying an increase in the amount of a biochemically functional conformation of the protein, which method may be in vitro or in vivo. Any of the types of cell recited herein may be used and these may contain any of the recited mutations, just as with the other methods of the invention.

As described herein, the opsin-binding agents useful in the methods of the invention and/or identified by any of the screening assays of the invention are available for use alone or in combination with one or more additional compounds to treat or prevent conditions associated with the wet or dry form of macular degeneration, retinitis pigmentosa, a retinal or macular dystrophy, Stargardt's disease, Sorsby's dystrophy, autosomal dominant drusen, Best's dystrophy, peripherin mutation associate with macular dystrophy, dominant form of Stargart's disease, North Carolina macular dystrophy, light toxicity (e.g., due to retinal surgery), retinitis pigmentosa or another ophthalmic condition related to the expression of a misfolded or mislocalized opsin protein. In one embodiment, an opsin-hinding compound of the invention (e.g., a non-retinoid or a retinoid that fails to covalently bind to opsin) is administered to a subject identified as having or at risk of developing such a condition. Optionally, the opsin binding agent is administered together with another therapeutic agent. In another embodiment, a non-retinoid opsin-binding compound of the invention is used in combination with a synthetic retinoid (e.g., as disclosed in U.S. Patent Publication No. 2004-0242704), and optionally with another active compound (e.g., as discussed herein). In still another exemplary embodiment, an opsin-binding compound is administered in combination with the proteasomal inhibitor MG132, the autophagy inhibitor 3-methyladenine, a lysosomal inhibitor, such as ammonium chloride, the ER-Golgi transport inhibitor brefeldin A, the Hsp90 chaperone inhibitor Geldamycin, the heat shock response activator Celastrol, the glycosidase inhibitor, and/or the histone deacetylase inhibitor Scriptaid, or any other agent that can stabilize a mutant P23H opsin protein in a biochemically functional conformation that allows it to associate with 11-cis-retinal to form rhodopsin.

Proteasomal Inhibitors

The 268 proteasome is a multicatalytic protease that cleaves ubiquinated proteins into short peptides. MG-132 is one proteasomal inhibitor that may be used. MG-132 is particularly useful for the treatment of retinitis pigmentosa and other ocular diseases related to protein aggregation or protein misfolding. Other proteasomal inhibitors useful in the methods of the invention include lactocystin (LC), clasto-lactocystin-beta-lactone, PSI (N-carbobenzoyl-IIe-Glu-(OtBu)-Ala-Leu-CHO), MG-132 (N-carbobenzoyl-Leu-Leu-Leu-CHO), MG-115 (N-carbobenzoyl-Leu-Leu-Nva-CHO), MG-101 (N-Acetyl-Leu-Leu-norLeu-CHO), ALLM (N-Acetyl-Leu-Leu-Met-CHO), N-carbobenzoyl-Gly-Pro-Phe-leu-CHO, N-carbobenzoyl-Gly-Pro-Ala-Phe-CHO, N-carbobenzoyl-Leu-Leu-Phe-CHO, and salts or analogs thereof. Other proteasomal inhibitors and their uses are described in U.S. Pat. No. 6,492,333.

Autophagy Inhibitors

Autophagy is an evolutionarily conserved mechanism for the degradation of cellular components in the cytoplasm, and serves as a cell survival mechanism in starving cells. During autophagy pieces of cytoplasm become encapsulated by cellular membranes, forming autophagic vacuoles that eventually fuse with lysosomes to have their contents degraded. Autophagy inhibitors may be used in combination with an opsin-binding or opsin-stabilizing compound. Autophagy inhibitors useful in the methods of the invention include, but are not limited to, 3-methyladenine, 3-methyl adenosine, adenosine, okadaic acid, N⁶-mercaptopurine riboside (N⁶-MPR), an aminothiolated adenosine analog, 5-amino-4-imidazole carboxamide riboside (AICAR), bafilomycin A1, and salts or analogs thereof.

Lysosomal Inhibitors

The lysosome is a major site of cellular protein degradation. Degradation of proteins entering the cell by receptor-mediated endocytosis or by pinocytosis, and of plasma membrane proteins takes place in lysosomes. Lysosomal inhibitors, such as ammonium chloride, leupeptin, trans-epoxysaccinyl-L-leucylamide-(4-guanidino) butane, L-methionine methyl ester, ammonium chloride, methylamine, chloroquine, and salts or analogs thereof, are useful in combination with an opsin-binding or opsin-stabilizing compound.

ER-Golgi Transport Inhibitors

Newly synthesized proteins enter the biosynthetic-secretory pathway in the endoplasmic reticulum (ER). To exit from the ER, the proteins must be properly folded. Those proteins that are misfolded are retained in the ER. Brefeldin A is one exemplary ER-Golgi transport inhibitor that is useful in combination with an opsin-binding or opsin-stabilizing compound in the methods of the invention.

HSP90 Chaperone Inhibitors

Heat shock protein 90 (Hsp90) is responsible for chaperoning proteins involved in cell signaling, proliferation and survival, and is essential for the conformational stability and function of a number of proteins. HSP-90 inhibitors are useful in combination with an opsin-binding or opsin-stabilizing compound in the methods of the invention. HSP-90 inhibitors include benzoquinone ansamycin antibiotics, such as geldanamycin and 17-allylamino-17-demethoxygeldanamycin (I7-AAG), which specifically bind to Hsp90, alter its function, and promote the proteolytic degradation of substrate proteins. Other HSP-90 inhibitors include, but are not limited to, radicicol, novobiocin, and any Hsp9O inhibitor that binds to the Hsp90 ATP/ADP pocket.

Heat Shock Response Activators

Celastrol, a quinone metbide triterpene, activates the human heat shock response. In combination with an opsin-binding or opsin-stabilizing compound, celastrol and other heat shock response activators are useful for the treatment of a protein conformation disease (PCD) Heat shock response activators include, but are not limited to, celastrol, celastrol methyl ester, dihydrocelastrol diacetate, celastrol butyl ester, dihydrocelastrol, and salts or analogs thereof.

Histone Deacetylase Inhibitors

Regulation of gene expression is mediated by several mechanisms, including the post-10 translational modifications of histones by dynamic acetylation and deacetylation. The enzymes responsible for reversible acetylationl/deacetylation processes are histone acetyltransferases (HATs) and histone deacetylases (HDACs), respectively. Histone deacetylase inhibitors include Scriptaid, APHA Compound 8, Apicidin, sodium butyrate, (−)-Depudecin, Sirtinol, trichostatin A, and salts or analogs thereof.

Glycosidase Inhibitors

Glycosidase inhibitors are one class of compounds that are useful in the methods of the invention, when administered in combination with an opsin-binding or opsin-stabilizing compound. Castanospermine, a polyhydroxy alkaloid isolated from plant sources, inhibits enzymatic glycoside hydrolysis. Castanospermine and its derivatives are particularly useful for the treatment of a protein conformation disorder, such as retinitis pigmentosa. Also useful in the methods of the invention are other glycosidase inhibitors, including australine hydrochloride, 6-Acetamido-6-deoxy-castanosperrnine, which is a powerful inhibitor of hexosaminidases, Deoxyfuconojirimycin hydrochloride (DFJ7), Deoxynojirimycin (DNJ), which inhibits glucosidase I and II, Deoxygalactonojirimycin hydrochloride (DGJ), which inhibits α-D-galactosidase, Deoxymannojirimycin hydrochloride (DM1), 2R,5R-Bis(hydroxymethyl)-3R,4R-dihydroxypyrrolidine (DMDP), also known as 2,5-dideoxy-2,5-imino-D-mannitol, 1,4-Dideoxy-1,4-imino-D-mannitol hydrochloride, (3R,4R,5R,6R)-3,4,5,6-Tetrahydroxyazepane Hydrochloride, which inhibits b-N-acetylglucosaminidase, 1,5-Dideoxy-1,5-imino-xylitol, which inhibits β-glucosidase, and Kifunensine, an inhibitor of mannosidase 1. Also useful in combination with an opsin-binding or opsin-stabilizing compound are N-butyldeoxynojirimycin (EDNJ), N-nonyl DNJ (NDND, N-hexyl DNJ (15TDNJ), N-methyldeoxynojirimycin (MDNJ), and other glycosidase inhibitors known in the art. Glycosidase inhibitors are available commercially, for example, from Industrial Research Limited (Wellington, New Zealand) and methods of using them are described, for example, in U.S. Pat. Nos. 4,894,388, 5,043,273, 5,103,008, 5,844,102, and 6,831,176; and in U.S. Patent Publication Nos. 20020006909.

Stabilization of Mutant Opsins

Retinitis pigmentosa is associated with the misfolding of an opsin (e.g., P23H opsin) (GenBank Accession Nos. NM_{—}000539 and NP_{—}000530), as well as with mutations in carbonic anhydrase IV (CA4)) (GenBank Accession Nos. NM_{—}000717 and NP_{—}000708) (Rebello et al., Proc Natl Acad Sci USA. 2004 Apr. 27; 101(17):6617-22). Compositions of the invention that increase the amount of opsin (e.g., P23H opsin) in a biochemically functional conformation are useful for the treatment of retinitis pigmentosa and other protein conformation disorders associated with mutations in the opsin polypeptide

One aspect is a method of treating a subject suffering from or susceptible to an ocular protein conformation disease or disorder, or symptom thereof. The method includes the step of administering to the subject a therapeutic amount of a compound herein sufficient to treat the disease or disorder or symptom thereof under conditions such that the disease or disorder or symptom thereof is treated. In certain embodiments, the disease or disorder is the wet or dry form of macular degeneration, retinitis pigmentosa, a retinal or macular dystrophy, Stargardt's disease, Sorsby's dystrophy, autosomal dominant drusen, Best's dystrophy, peripherin mutation associated with macular dystrophy, dominant form of Stargart's disease, North Carolina macular dystrophy, light toxicity (e.g., due to retinal surgery), or retinitis pigmentosa. In certain preferred embodiments, the subject is a human. In certain preferred embodiments, the subject is a subject identified as being in need of such treatment. In certain embodiments, the method includes administration of an additional therapeutic agent.

In certain embodiments, the method further includes the step of determining a level of Marker (e.g., wild-type or misfolded opsin or rhodopsin) in the subject. In certain embodiments, the step of determining of the level of Marker is performed prior to administration of the compound of the formulae hereinto the subject. In certain embodiments, the determining of the level of Marker is performed subsequent to administration of the compound of the formulae hereinto the subject. In certain embodiments, the determining of the level of Marker is performed prior to and subsequent to administration of the compound of the formulae hereinto the subject. In certain embodiments, the levels of Marker performed prior to and subsequent to administration of the compound of the formulae hereinto the subject are compared. In certain embodiments, the comparison of Marker levels is reported by a clinic, laboratory, or hospital agent to a health care professional. In certain embodiments, when the level of Marker performed prior to administration of the compound of the formulae hereinto the subject is lower or higher (depending on the Marker) than the level of Marker performed subsequent to administration of the compound of the formulae hereinto the subject, then the amount of compound administered to the subject is an effective amount.

In another aspect, an embodiment provides kits for treatment of a disease(s) or disorder(s) or symptoms thereof, including ocular protein conformation diseases, such as the wet or dry form of macular degeneration, retinitis pigmentosa, a retinal or macular dystrophy, Stargardt's disease, Sorsby's dystrophy, autosomal dominant drusen, Best's dystrophy, peripherin mutation associated with macular dystrophy, dominant form of Stargart's disease, North Carolina macular dystrophy, light toxicity (e.g., due to retinal surgery), or diabetic retinopathy. In one embodiment, the kit includes an effective amount of a compound of the formulae herein in unit dosage form, together with instructions for administering the compound of the formulae hereinto a subject suffering from or susceptible to a disease or disorder or symptoms thereof, including those of a cardiovascular nature. In preferred embodiments, the compound of the formulae herein is a therapeutic compound progenitor.

In another aspect, an embodiment provides a method of treating a mammal to correct opsin protein conformation or localization or to treat an ocular protein conformation disease, such as the wet or dry form of macular degeneration, retinitis pigmentosa, a retinal or macular dystrophy, Stargardt's disease, Sorsby's dystrophy, autosomal dominant drusen, Best's dystrophy, peripherin mutation associate with macular dystrophy, dominant form of Stargart's disease, North Carolina macular dystrophy, light toxicity (e.g., due to retinal surgery), and diabetic retinopathy, the method including administering to the mammal a therapeutically effective amount of at least one compound of the invention (e.g., a compound of any of the formulae herein) capable of binding to opsin at or near the opsin binding pocket.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

Another aspect is a method of making a pharmaceutical composition delineated herein, including the step of combining a compound herein (e.g., a compound of any of the formulae herein) with a pharmaceutically acceptable carrier. The method can further include combining an additional therapeutic agent with the compound and/or carrier. Compounds (or salts or solvates thereof) of the invention include 1-(3,5-dimethyl-1H-pyrazol-4-yl)-ethanone, 1-furan-2-ylmethyl-2,4-dioxo-1,2,3,4-tetrahydro-pyrimidine-5-carbonitrile, phenyl-phosphinic acid, 2-methyl-4-nitro-pyridine, 3,6-bis-(2-hydroxyethy)-piperazine-2,5-dione, diisopropylaminoacetonitrile, 3,4-methylenedioxybenzonitrile, diethyl(2-mercaptoethyl)amine, 6-imino-1-methyl-1,6-dihydro-3-pyridinecarboxamide, 1H-1,2,3-benzotriazol-1-amine, 4-salicylideneamino-1,2,4-triazole, β-ionone, and cis-1,3-dimethylcyclohexane that are representative embodiments of the formulae herein and are useful in the methods delineated herein.

The compounds, compositions, methods, and kits of the invention are useful for the treatment of conditions such as diabetic retinopathy, wet or dry form of macular degeneration, retinitis pigmentosa, a retinal or macular dystrophy, Stargardt's disease, Sorsby's dystrophy, autosomal dominant drusen, Best's dystrophy, peripherin mutation associate with macular dystrophy, dominant form of Stargart's disease, North Carolina macular dystrophy, light toxicity (e.g., due to retinal surgery), or retinitis pigmentosa.

Pharmaceutical Compositions

The present invention features pharmaceutical preparations comprising compounds together with pharmaceutically acceptable carriers, where the compounds provide for the generation of a mutant protein in a biochemically functional conformation. Such preparations have both therapeutic and prophylactic applications. In one embodiment, a pharmaceutical composition includes an opsin-binding or opsin-stabilizing compound (e.g., a compound of Table 1 or Table 2, or β-ionone or Cis-1,3-dimethylcyclohexane) or a pharmaceutically acceptable salt thereof; optionally in combination with at least one additional compound that is a proteasomal inhibitor, an autophagy inhibitor, a lysosomal inhibitor, an inhibitor of protein transport from the ER to the Golgi, an Hsp9O chaperone inhibitor, a heat shock response activator, a glycosidase inhibitor, or a histone deacetylase inhibitor. The opsin-binding or opsin-stabilizing compound is preferably not a natural or synthetic retinoid. The opsin-binding or opsin-stabilizing compound and are formulated together or separately. Compounds of the invention may be administered as part of a pharmaceutical composition. The compositions should be sterile and contain a therapeutically effective amount of the opsin-binding or opsin-stabilizing compound in a unit of weight or volume suitable for administration to a subject. The compositions and combinations of the invention can be part of a pharmaceutical pack, where each of the compounds is present in individual dosage amounts.

The phrase “pharmaceutically acceptable” refers to those compound of the inventions of the present invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutical compositions of the invention to be used for prophylactic or therapeutic administration should be sterile. Sterility is readily accomplished by filtration through sterile filtration membranes (e.g., 0.2 μm membranes), by gamma irradiation, or any other suitable means known to those skilled in the art. Therapeutic opsin-binding or opsin-stabilizing compound compositions generally are placed into a container having a sterile access port, for example, an intravenous solution bag or vial having a stopper pierceable by a hypodermic injection needle. These compositions ordinarily will be stored in unit or multi-dose containers, for example, sealed ampoules or vials, as an aqueous solution or as a lyophilized formulation for reconstitution. The compounds may be combined, optionally, with a pharmaceutically acceptable excipient.

The components of the pharmaceutical compositions also are capable of being co-mingled with the molecules of the present invention, and with each other, in a manner such that there is no interaction that would substantially impair the desired pharmaceutical efficacy.

Compounds of the present invention can be contained in a pharmaceutically acceptable excipient. The excipient preferably contains minor amounts of additives such as substances that enhance isotonicity and chemical stability. Such materials are non-toxic to recipients at the dosages and concentrations employed, and include buffers, such as phosphate, citrate, succinate, acetate, lactate, tartrate, and other organic acids or their salts; tris-hydroxymethylaminomethane (TRIS), bicarbonate, carbonate, and other organic bases and their salts; antioxidants, such as ascorbic acid; low molecular weight (for example, less than about ten residues) polypeptides, e.g., polyarginine, polylysine, polyglutamate and polyaspartate; proteins, such as serum albumin, gelatin, or immunoglobulins; hydrophilic polymers, such as polyvinylpyrrolidone (PVP), polypropylene glycols (PPGs), and polyethylene glycols (PEGs); amino acids, such as glycine, glutamic acid, aspartic acid, histidine, lysine, or arginine; monosaccharides, disaccharides, and other carbohydrates including cellulose or its derivatives, glucose, mannose, sucrose, dextrins or sulfated carbohydrate derivatives, such as heparin, chondroitin sulfate or dextran sulfate; polyvalent metal ions, such as divalent metal ions including calcium ions, magnesium ions and manganese ions; chelating agents, such as ethylenediamine tetraacetic acid (EDTA); sugar alcohols, such as mannitol or sorbitol; counterions, such as sodium or ammonium; and/or nonionic surfactants, such as polysorbates or poloxamers. Other additives may be included, such as stabilizers, anti-microbials, inert gases, fluid and nutrient replenishers (i.e., Ringer's dextrose), electrolyte replenishers, and the like, which can be present in conventional amounts.

The compositions, as described above, can be administered in effective amounts. The effective amount will depend upon the mode or administration, the particular condition being treated and the desired outcome. It may also depend upon the stage of the condition, the age and physical condition of the subject, the nature of concurrent therapy, if any, and like factors well known to the medical practitioner. For therapeutic applications, it is that amount sufficient to achieve a medically desirable result.

With respect to a subject suffering from retinitis pigmentosa, an effective amount is sufficient to increase the level of a correctly folded opsin protein in a cell. With respect to a subject having a disease or disorder related to a misfolded protein, an effective amount is an amount sufficient to stabilize, slow, or reduce the a symptom associated with a pathology such as retinitis pigmentosa. Generally, doses of the compounds of the present invention would be from about 0.01 mg/kg per day to about 1000 mg/kg (e.g., 0.01, 0.05, 0.1, 0.25, 0.5, 1.0, 5, 10, 15, 20, 25) per day. It is expected that doses ranging from about 50 to about 2000 mg/kg (e.g., 50, 100, 200, 250, 500, 750, 1000, 1250, 1500, 1750, 2000) will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of a composition of the present invention.

A variety of administration routes are available. The methods of the invention, generally speaking, may be practiced using any mode of administration that is medically acceptable, meaning any mode that produces effective levels of the active compounds without causing clinically unacceptable adverse effects. In one preferred embodiment, a composition of the invention is administered intraocularly. Other modes of administration include oral, rectal, topical, intraocular, buccal, intravaginal, intracisternal, intracerebroventricular, intratracheal, nasal, transdermal, within/on implants, or parenteral routes. Compositions comprising a composition of the invention can be added to a physiological fluid, such as to the intravitreal humor. For CNS administration, a variety of techniques are available for promoting transfer of the therapeutic across the blood brain barrier including disruption by surgery or injection, drugs which transiently open adhesion contact between the CNS vasculature endothelial cells, and compounds that facilitate translocation through such cells. Oral administration can be preferred for prophylactic treatment because of the convenience to the patient as well as the dosing schedule.

Pharmaceutical compositions of the invention can optionally further contain one or more additional proteins as desired, including plasma proteins, proteases, and other biological material, so long as it does not cause adverse effects upon administration to a subject. Suitable proteins or biological material may be obtained from human or mammalian plasma by any of the purification methods known and available to those skilled in the art; from supernatants, extracts, or lysates of recombinant tissue culture, viruses, yeast, bacteria, or the like that contain a gene that expresses a human or mammalian plasma protein which has been introduced according to standard recombinant DNA techniques; or from the fluids (e.g., blood, milk, lymph, urine or the like) or transgenic animals that contain a gene that expresses a human plasma protein which has been introduced according to standard transgenic techniques.

Pharmaceutical compositions of the invention can comprise one or more ph buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions of the invention can also contain one or more osmotic modulating agents, i.e., a compound that modulates the osmotic properties (e.g., tonicity, osmolality and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) maybe present in any concentration sufficient to modulate the osmotic properties of the formulation.

Compositions comprising an opsin-binding or opsin-stabilizing compound of the present invention can contain multivalent metal ions, such as calcium ions, magnesium ions and/or manganese ions. Any multivalent metal ion that helps stabilizes the composition and that will not adversely affect recipient individuals may be used. The skilled artisan, based on these two criteria, can determine suitable metal ions empirically and suitable sources of such metal ions are known, and include inorganic and organic salts.

Pharmaceutical compositions of the invention can also be a non-aqueous liquid formulation. Any suitable non-aqueous liquid may be employed, provided that it provides stability to the active agents (a) contained therein. Preferably, the non-aqueous liquid is a hydrophilic liquid. illustrative examples of suitable non-aqueous liquids include: glycerol; dimethyl sulfoxide (DMSO); polydimethylsiloxane (PMS); ethylene glycols, such as ethylene glycol, diethylene glycol, triethylene glycol, polyethylene glycol (“PEG”) 200, PEG 300, and PEG 400; and propylene glycols, such as dipropylene glycol, tripropylene glycol, polypropylene glycol (“PPG”) 425, PPG 725, PPG 1000, PEG 2000, PEG 3000 and PEG 4000.

Pharmaceutical compositions of the invention can also be a mixed aqueous/non-aqueous liquid formulation. Any suitable non-aqueous liquid formulation, such as those described above, can be employed along with any aqueous liquid formulation, such as those described above, provided that the mixed aqueous/non-aqueous liquid formulation provides stability to the compound contained therein. Preferably, the non-aqueous liquid in such a formulation is a hydrophilic liquid. Illustrative examples of suitable non-aqueous liquids include: glycerol; DMSO; EMS; ethylene glycols, such as PEG 200, PEG 300, and PEG 400; and propylene glycols, such as PPG 425, PPG 725, PEG 1000, PEG 2000, PEG 3000 and PEG 4000. Suitable stable formulations can permit storage of the active agents in a frozen or an unfrozen liquid state. Stable liquid formulations can be stored at a temperature of at least −70° C., but can also be stored at higher temperatures of at least 0° C., or between about 0.1° C. and about 42° C., depending on the properties of the composition. It is generally known to the skilled artisan that proteins and polypeptides are sensitive to changes in pH, temperature, and a multiplicity of other factors that may affect therapeutic efficacy.

In certain embodiments a desirable route of administration can be by pulmonary aerosol. Techniques for preparing aerosol delivery systems containing polypeptides are well known to those of skill in the art. Generally, such systems should utilize components that will not significantly impair the biological properties of the antibodies, such as the paratope binding capacity (see, for example, Sciarra and Cutie, “Aerosols,” in Reminqton's Pharmaceutical Sciences 18th edition, 1990, pp 1694-1712; incorporated by reference). Those of skill in the art can readily modify the various parameters and conditions for producing polypeptide aerosols without resorting to undue experimentation.

Other delivery systems can include time-release, delayed release or sustained release delivery systems. Such systems can avoid repeated administrations of compositions of the invention, increasing convenience to the subject and the physician. Many types of release delivery systems are available and known to those of ordinary skill in the art. They include polymer base systems such as polylactides (U.S. Pat. No. 3,773,919; European Patent No. 58,481), poly(lactide-glycolide), copolyoxalates polycaprolactones, polyesteramides, polyorthoesters, polyhydroxybutyric acids, such as poly-D-(−)-3-hydroxybutyric acid (European Patent No. 133,988), copolymers of L-glutamic acid and gamma-ethyl-L-glutamate (Sidman, K R. et at, Biopolymers 22: 547-556), poly (2-hydroxyethyl methacrylate) or ethylene vinyl acetate (Langer, ft et at, J. Biomed. Mater. Res. 15:267-277; Langer, B. Chem. Tech. 12:98-105), and polyanhydrides.

Other examples of sustained-release compositions include semi-permeable polymer matrices in the form of shaped articles, e.g., films, or microcapsules. Delivery systems also include non-polymer systems that are: lipids including sterols such as cholesterol, cholesterol esters and fatty acids or neutral fats such as mono-, di- and tri-glycerides; hydrogel release systems such as biologically-derived bioresorbable hydrogel (i.e., chitin hydrogels or chitosan hydrogels); sylastic systems; peptide based systems; wax coatings; compressed tablets using conventional binders and excipients; partially fined implants; and the like. Specific examples include, but are not limited to: (a) erosional systems in which the agent is contained in a form within a matrix, such as those described in 13.5. U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480.

Another type of delivery system that can be used with the methods and compositions of the invention is a colloidal dispersion system. Colloidal dispersion systems include lipid-based systems including oil-in-water emulsions, micelles, mixed micelles, and liposomes. Liposomes are artificial membrane vessels, which are useful as a delivery vector in vivo or in vitro. Large unilamellar vessels (LUV), which range in size from 0.2-4.0 μm, can encapsulate large macromolecules within the aqueous interior and be delivered to cells in a biologically active form (Fraley, R., and Papahadjopoulos, D., Trends Biochem. Sci. 6: 77-80).

Liposomes can be targeted to a particular tissue by coupling the liposome to a specific ligand such as a monoclonal antibody, sugar, glycolipid, or protein. Liposomes are commercially available from Gibco BRL, for example, as LIPOFECTIN™ and LIPOFECTACE™, which are formed of cationic lipids such as N-[1-(2,3dioleyloxy)-propyl]-N,N,N-trimethylammonium chloride (DOTMA) and dimethyl dioctadecylammonium bromide (DDAB). Methods for making liposomes are well known in the art and have been described in many publications, for example, in DE 3,218,121; Epstein et al., Proc. Nail. Acad. Sci. (USA) 82:3688-3692 (1985); I-Twang et al., Proc. Natl, Acad. Sci. (USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Liposomes also have been reviewed by Gregoriadis, G., Trends Biotechnol., 3: 235-241).

Another type of vehicle is a biocompatible microparticle or implant that is suitable for implantation into the mammalian recipient. Exemplary bioerodible implants that are useful in accordance with this method are described in PCT International application no. PCTIUS/03307 (Publication No-WO 95/24929, entitled “Polymeric Gene Delivery System”). PCT/US/0307 describes biocompatible, preferably biodegradable polymeric matrices for containing an exogenous gene under the control of an appropriate promoter. The polymeric matrices can be used to achieve sustained release of the exogenous gene or gene product in the subject.

The polymeric matrix preferably is in the form of a microparticle, such as a microsphere (wherein an agent is dispersed Throughout a solid polymeric matrix) or a microcapsule (wherein an agent is stored in the core of a polymeric shell). Microcapsules of the foregoing polymers containing drugs are described in, for example, U.S. Pat. No. 5,075,109. Other forms of the polymeric matrix for containing an agent include films, coatings, gels, implants, and stents. The size and composition of the polymeric matrix device is selected to result in favorable release kinetics in the tissue into which the matrix is introduced. The size of the polymeric matrix further is selected according to the method of delivery that is to be used. Preferably, when an aerosol route is used the polymeric matrix and composition are encompassed in a surfactant vehicle. The polymeric matrix composition can be selected to have both favorable degradation rates and also to be formed of a material, which is a bioadhesive, to further increase the effectiveness of transfer. The matrix composition also can be selected not to degrade, but rather to release by diffusion over an extended period of time, The delivery system can also be a biocompatible microsphere that is suitable for local, site-specific delivery. Such microspheres are disclosed in Chickering, D. B., et al., Biotechnol. Bioeng, 52: 96-101; Mathiowitz, B., et at., Nature 386: 410-414.

Both non-biodegradable and biodegradable polymeric matrices can be used to deliver the compositions of the invention to the subject. Such polymers may be natural or synthetic polymers. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

Exemplary synthetic polymers which can be used to form the biodegradable delivery system include: polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulfate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene, polyvinylpyrrolidone, and polymers of lactic acid and glycolic acid, polyanhydrides, poly(ortho)esters, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Methods of Ocular Delivery

The compositions of the invention are particularly suitable for treating ocular diseases or conditions, such as retinitis pigmentosa.

In one approach, the compositions of the invention are administered through an ocular device suitable for direct implantation into the vitreous of the eye. The compositions of the invention may be provided in sustained release compositions, such as those described in, for example, U.S. Pat. Nos. 5,672,659 and 5,595,760. Such devices are found to provide sustained controlled release of various compositions to treat the eye without risk of detrimental local and systemic side effects. An object of the present ocular method of delivery is to maximize the amount of drug contained in an intraocular device or implant while minimizing its size in order to prolong the duration of the implant. See, e.g., U.S. Pat. Nos. 5,378,475; 6,375,972, and 6,756,058 and U.S. Publications 20050096290 and 200501269448. Such implants may be biodegradable and/or biocompatible implants, or may be non-biodegradable implants.

Biodegradable ocular implants are described, for example, in U.S. Patent Publication No. 20050048099. The implants may be permeable or impermeable to the active agent, and may be inserted into a chamber of the eye, such as the anterior or posterior chambers or may be implanted in the sclera, transchoroidal space, or an avascularized region exterior to the vitreous. Alternatively, a contact lens that acts as a depot for compositions of the invention may also be used for drug delivery.

In a preferred embodiment, the implant may be positioned over an avascular region, such as on the sclera, so as to allow for transcleral diffusion of the drug to the desired site of treatment, e.g. the intraocular space and macula of the eye. Furthermore, the site of transcleral diffusion is preferably in proximity to the macula. Examples of implants for delivery of a composition of the invention include, but are not limited to, the devices described in U.S. Pat. Nos. 3,416,530; 3,828,777; 4,014,335; 4,300,557; 4,327,725; 4,853,224; 4,946,450; 4,997,652; 5,147,647; 164,188; 5,178,635; 5,300,114; 5,322,691; 5,403,901; 5,443,505; 5,466,466; 5,476,511; 5,516,522; 5,632,984; 5,679,666; 5,710,165; 5,725,493; 5,743,274; 5,766,242; 5,766,619; 5,770,592; 5,773,019; 5,824,072; 5,824,073; 5,830,173; 5,836,935; 5,869,079, 5,902,598; 5,904,144; 5,916,584; 6,001,386; 6,074,661; 6,110,485; 6,126,687; 6,146.366; 6,251,090; and 6,299,895, and in WO 01/30323 and WO 01/28474, all of which are incorporated herein by reference.

Examples include, but are not limited to the following: a sustained release drug delivery system comprising an inner reservoir comprising an effective amount of an agent effective in obtaining a desired local or systemic physiological or pharmacological effect, an inner tube impermeable to the passage of the agent, the inner tube having first and second ends and covering at least a portion of the inner reservoir, the inner tube sized and formed of a material so that the inner tube is capable of supporting its own weight, an impermeable member positioned at the inner tube first end, the impermeable member preventing passage of the agent out of the reservoir through the inner tube first end, and a permeable member positioned at the inner tube second end, the permeable member allowing diffusion of the agent out of the reservoir through the inner tube second end; a method for administering a compound of the invention to a segment of an eye, the method comprising the step of implanting a sustained release device to deliver the compound of the invention to the vitreous of the eye or an implantable, sustained release device for administering a compound of the invention to a segment of an eye; a sustained release drug delivery device comprising: a) a drug core comprising a therapeutically effective amount of at least one first agent effective in obtaining a diagnostic effect or effective in obtaining a desired local or systemic physiological or pharmacological effect; b) at least one unitary cup essentially impermeable to the passage of the agent that surrounds and defines an internal compartment to accept the drug core, the unitary cup comprising an open top end with at least one recessed groove around at least some portion of the open top end of the unitary cup; c) a permeable plug which is permeable to the passage of the agent, the permeable plug is positioned at the open top end of the unitary cup wherein the groove interacts with the permeable plug holding it in position and closing the open top end, the permeable plug allowing passage of the agent out of the drug core, though the permeable plug, and out the open top end of the unitary cup; and d) at least one second agent effective in obtaining a diagnostic effect or effective in obtaining a desired local or systemic physiological or pharmacological effect; or a sustained release drug delivery device comprising: an inner core comprising an effective amount of an agent having a desired solubility and a polymer coating layer, the polymer layer being permeable to the agent, wherein the polymer coating layer completely covers the inner core.

Other approaches for ocular delivery include the use of liposomes to target a compound of the present invention to the eye, and preferably to retinal pigment epithelial cells and/or Bruch's membrane. For example, the compound maybe complexed with liposomes in the manner described above, and this compound/liposome complex injected into patients with an ocular PCD, such as retinitis pigmentosa, using intravenous injection to direct the compound to the desired ocular tissue or cell. Directly injecting the liposome complex into the proximity of the retinal pigment epithelial cells or Bruch's membrane can also provide for targeting of the complex with some forms of ocular PCD, such as retinitis pigmentosa. In a specific embodiment, the compound is administered via intra-ocular sustained delivery (such as VITRASERT or ENVISION. In a specific embodiment, the compound is delivered by posterior subtenons injection. In another specific embodiment, microemulsion particles containing the compositions of the invention are delivered to ocular tissue to take up lipid from Bruchs membrane, retinal pigment epithelial cells, or both.

Nanoparticles are a colloidal carrier system that has been shown to improve the efficacy of the encapsulated drug by prolonging the serum half-life. Polyalkylcyanoacrylates (PACAs) nanoparticles are a polymer colloidal drug delivery system that is in clinical development, as described by Stella et al, J. Pharm. Sci., 2000. 89: p. 1452-1464; Brigger et al., Tnt. J. Pharm., 2001. 214: p. 37-42; Calvo et al., Pharm. Res., 2001. 18: p. 1157-1166; and Li et al., Biol. Pharm. Bull., 2001. 24: p. 662-665. Biodegradable poly (hydroxyl acids), such as the copolymers of poly (lactic acid) (PLA) and poly (lactic-co-glycolide) (PLGA) are being extensively used in biomedical applications and have received FDA approval for certain clinical applications. In addition, PEG-PLGA nanoparticles have many desirable carrier features including (i) that the agent to be encapsulated comprises a reasonably high weight fraction (loading) of the total carrier system; (ii) that the amount of agent used in the first step of the encapsulation process is incorporated into the final carrier (entrapment efficiency) at a reasonably high level; (iii) that the carrier have the ability to be freeze-dried and reconstituted in solution without aggregation; (iv) that the carrier be biodegradable; (v) that the carrier system be of small size; and (vi) that the carrier enhance the particles persistence.

Nanoparticles are synthesized using virtually any biodegradable shell known in the art. In one embodiment, a polymer, such as poly (lactic-acid) (PLA) or poly (lactic-co-glycolic acid) (PLGA) is used. Such polymers are biocompatible and biodegradable, and are subject to modifications that desirably increase the photochemical efficacy and circulation lifetime of the nanoparticle. in one embodiment, the polymer is modified with a terminal carboxylic acid group (COOH) that increases the negative charge of the particle and thus limits the interaction with negatively charge nucleic acid aptamers. Nanoparticles are also modified with polyethylene glycol (PEG), which also increases the half-life and stability of the particles in circulation. Alternatively, the COOH group is converted to an N-hydroxysuccinimide (NHS) ester for covalent conjugation to amine-modified aptamers.

Biocompatible polymers useful in the composition and methods of the invention include, but are not limited to, polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulfate sodium salt poly-methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), polyvinyl acetate, polyvinyl chloride polystyrene, polyvinylpryrrolidone, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutylmethacrylate), poly(hexylmethacrylate poly(isodecylmethacrylate), poly(lauryl methacrylate), polyphenyl methacrylate), poly(methyl acrylate), poly(isopropyl acrylate), poly(isobutyl acrylate), poly(octadecyl acrylate) and combinations of any of these, In one embodiment, the nanoparticles of the invention include PEG-PLGA polymers.

Compositions of the invention may also be delivered topically. For topical delivery, the compositions of the invention are provided in any pharmaceutically acceptable excipient that is approved for ocular delivery. Preferably, the composition is delivered in drop form to the surface of the eye. For some applications, the delivery of the composition relies on the diffusion of the compounds through the cornea to the interior of the eye.

Those of skill in the art will recognize that the best treatment regimens for using any of the compounds of the present invention to treat retinitis pigmentosa can be straightforwardly determined. This is not a question of experimentation, but rather one of optimization, which is routinely conducted in the medical arts. In vivo studies in nude mice often provide a starting point from which to begin to optimize the dosage and delivery regimes. The frequency of injection will initially be once a week, as has been done in some mice studies. However, this frequency might be optimally adjusted from one day to every two weeks to monthly, depending upon the results obtained front the initial clinical trials and the needs of a particular patient.

Human dosage amounts can initially be determined by extrapolating from the amount of compound used in mice, as a skilled artisan recognizes it is routine in the art to modify the dosage for humans compared to animal models. fin certain embodiments it is envisioned that the dosage may vary from between about 1 mg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose maybe about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that higher does may be used, such doses may be in the range of about 5 mg compound/Kg body to about 20 mg compound/Kg body. In other embodiments the doses may be about 8, 10, 12, 14, 16 15 or 18 mg/Kg body weight. Of course, this dosage amount may be adjusted upward or downward, as is routinely done in such treatment protocols, depending on the results of the initial clinical trials and the needs of a particular patient.

Screening Assays

As discussed herein, useful compounds correct or prevent protein mis-folding by increasing the amount of a mutant protein that is in a biochemically active conformation. Any number of methods are available for carrying out screening assays to identify such compounds. In one approach, a mutant protein that fails to adopt a wild-type protein conformation is expressed in a cell (e.g., a cell in vitro or in vivo) the cell is contacted with a candidate compound; and the effect of the compound on the conformation of the mutant protein is assayed using any method known in the art or described herein. A compound that increases the yield of correctly folded protein present in the contacted cell relative to a control cell that was not contacted with the compound, is considered useful in the methods of the invention. An increase in the amount of a correctly folded protein is assayed, for example, by measuring the protein's absorption at a characteristic wavelength (e.g., 498 nm for rhodopsin), by measuring a decrease in intracellular protein aggregation by measuring a decrease in cytotoxicity, by measuring the mitigation of an RP-related phenotype, or by measuring an increase in the biological activity of the protein using any standard method (e.g., enzymatic activity association with a ligand). In another embodiment, a candidate compound is identified as useful in the methods of the invention by a screening assay that that increases the total yield of opsin present in a contacted cell relative to the amount present in an untreated control cell. In another embodiment, the compound increases visual function assayed using an electroretinogram (ERG) relative to the visual function in an untreated control animal. In another embodiment, the compound reduces opsin mislocalization or increases correctly localized opsin (i.e., opsin that is localized to a photoreceptor membrane) relative to the localization of opsin in an untreated control cell. In yet another embodiment, the compound improves retinal morphology or retinal preservation in a histological assay in a contacted animal relative to an untreated control animal.

Useful compounds increase the amount of protein in a biochemically functional conformation by at least 10%, 15%, or 20%, or preferably by 25%, 50%, or 75%; or most preferably by at least 100%, 200%, 300% or even 400%.

In general, the compounds identified by the present invention, as well as disclosed herein, have a number of advantages over anything available in the art. The compounds useful in the methods of the invention are non-retinoid compounds. This is important because the level of retinoids (such as 11-cis-retinal) entering the eye is tightly controlled and larger than acceptable doses wind up be sequestered in, for example, the RPE cells and most of the retinoid does not make it to the rod cells. In addition, they are structurally diverse and do not covalently bind to opsin so that once the mis-folded opsin has been conformationally corrected and inserted into the membrane of a rod cell the compounds of the invention can dissociate and permit 11-cis-retinal to bind and form rhodopsin. Because the compounds of the invention bind inside the retinal binding pocket of opsin, they prevent simultaneous binding of 11-cis-retinal before the conformationally correct opsin is inserted properly into the cell membrane. In addition, the compounds of the invention can bind to mis-folded opsin when it is initially formed in the endoplasmic reticulum, thereby acting as a pharmacological chaperone for directing the opsin toward the cell membrane. Unlike previously used chemical chaperones and pharmacological chaperones, the compounds of the invention are selective for opsin and are not retinoid derivatives.

The compounds useful in the present invention may show several types of activity that can be readily screened for. Most importantly, the compounds of the invention are non-retinoids showing the ability to reversibly bind to opsin protein to prevent binding of physiological retinoids, such as 11-cis-retinal, to the opsin molecule. Such binding commonly ties up the retinal binding pocket of the molecule. In a typical competition assay of the invention, a non-retinoid compound (i.e., not tightly regulated by the retina as to amount entering rod cells) is sought that reversibly competes with 11-cis-retinal. Over time this will slow the rate of formation of rhodopsin relative to the rate when 11-cis-retinal alone is present. Here, when the assay is conducted in the presence of 11-cis-retinal, the rate of formation of rhodopsin can be measured as a way of determining competition for the retinal binding pocket, for example, by determining the rate of increase in the 500 nm peak characteristic for rhodopsin. Cells producing a mutant opsin can also be studied in such assays so long as the opsin is conformationally correct.

Examination of the crystal structure for rhodopsin shows that the retinal binding pocket is only big enough for one molecule to be inside at a time so that when a compound of the invention is in the pocket then 11-cis-retinal cannot enter. Thus, compounds of the invention compete with 11-cis-retinal for a place at or in the retinal binding pocket, but do not bind covalently (like 11-cis-retinal does) so that the binding of the test compound is reversible. A useful compound will exhibit a rate of rhodopsin formation that is at least about 2 to 5 fold lower than that observed in the presence of 11-cis-retinal when said test compound is not present.

In a preferred embodiment, the compounds of the invention also act to increase opsin in the rod cell because the amount of opsin is affected by the amount of folded rhodopsin. Here, total opsin is the sum of folded and mis-folded protein. The mis-folded opsin, when contacted with a compound of the invention (for example, in the endoplasmic reticulum of the rod cell) is then properly folded, thereby leading to reduced degradation and turn-over so that the total opsin in the cell can be increased. Such compounds are readily identified using cultures of cells producing a mutant opsin (such as the P23H mutant) that allow detection of entry of the compound into the cell and the detection of increased opsin, especially increased conformationally correct opsin. This increase in opsin can be detected using opsin-specific antibodies and a dot blot assay (see Example 3).

In a further preferred embodiment, the compounds of the invention also increase folded protein without 11-cis-retinal being present (where the latter compound can also act as a pharmacological chaperone but with less activity than the compounds of the invention). To screen for this activity, cells producing wild type and mutated opsin were induced to produce opsin using tetracycline in the absence of 11-cis-retinal and the test compound added to the cell culture. After about 48 hours the cell were harvested and opsin production ceased. Addition of 11-cis-retinal resulted in production of rhodopsin (indicating presence of properly folded protein). The amount of this pigment formed was then assayed in the UV-VIS region as a 280/500 nm ratio (280 nm for protein total and 500 nm for pigment, i.e., rhodopsin). The compounds of the invention showed increased rhodopsin production relative to total protein (with test compound absent in the control) versus when only 11-cis-retinal was present as chaperone.

In another preferred embodiment, compounds of the invention act to correct visual problems in an animal model of retinitis pigmentosa. For example, we have used transgenic animals (all mice) producing mutant opsins (for a total of 3 different mutations, including P23H). Such animals are also known from the art. In all cases, the cellular phenotype is the same.

Test Compounds and Extracts

In general, compounds capable of increasing the amount of a correctly folded protein in a cell are identified from large libraries of either natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceanographic Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity in correcting a mis-folded protein should be employed whenever possible.

When a crude extract is found to correct the conformation of a mis-folded protein further fractionation of the positive lead extract is necessary to isolate chemical constituents responsible for the observed effect. Thus, the goal of the extraction, fractionation, and purification process is the careful characterization and identification of a chemical entity within the crude extract that increase the yield of a correctly folded protein. Methods of fractionation and purification of such heterogeneous extracts are known in the art. If desired, compounds shown to be useful agents for the treatment of any pathology related to a mis-folded protein or protein aggregation are chemically modified according to methods known in the art.

Combination Therapies

Compositions of the invention useful for the treatment of retinitis pigmentosa (or diabetes mellitus) can optionally be combined with additional therapies. For retinitis pigmentosa, standard therapies include vitamin A supplements.

Kits

The invention provides kits for the treatment or prevention of retinitis pigmentosa or symptoms thereof. In one embodiment, the kit includes a pharmaceutical pack comprising an effective amount of an opsin-binding or opsin-stabilizing compound. Preferably, the compositions arc present in unit dosage form. In some embodiments, the kit comprises a sterile container, which contains a therapeutic or prophylactic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments. In certain embodiments, the kit further comprises any one or more of The following compounds: a proteasomal inhibitor (e.g., MG132), an autophagy inhibitor (e.g., 3-methyladenine), a lysosomal inhibitor (e.g., ammonium chloride), an inhibitor of protein transport from the ER to the Golgi (e.g., brefeldin A), an Hsp90 chaperone inhibitor (e.g., Geldanamycin), a heat shock response activator (e.g., Celastrol), a glycosidase inhibitor (e.g., castanospermine) and a Histone deacetylase inhibitor (e.g., Scriptaid).

If desired compositions of the invention or combinations thereof are provided together with instructions for administering them to a subject having or at risk of developing retinitis pigmentosa. The instructions will generally include information about the use of the compounds for the treatment or prevention of retinitis pigmentosa. In other embodiments, the instructions include at least one of the following: description of the compound or combination of compounds; dosage schedule and administration for treatment of retinitis pigmentosa or symptoms thereof; precautions; warnings; indications; counter-indications; overdosage information; adverse reactions; animal pharmacology; clinical studies; and/or references. The instructions may be printed directly on the container (when present), or as a label applied to the container, or as a to separate sheet, pamphlet, card, or folder supplied in or with the container.

### EXAMPLES

In carrying out the procedures of the present invention it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

The invention is described in more detail in the following non-limiting examples. It is to be understood that these methods and examples in no way limit the invention to the embodiments described herein and that other embodiments and uses will no doubt suggest themselves to those skilled in the art.

Retinitis pigmentosa (RP) comprises a heterogeneous group of inherited retinal disorders that lead to rod photoreceptor death. The death of photoreceptors results in night blindness and subsequent tunnel vision due to the progressive loss of peripheral vision in patients suffering from retinitis pigmentosa. Between 20-25% of patients with Autosomal Dominant Retinitis Pigmentosa (ADRP) have a mutation in the rhodopsin gene, the most common mutation being P23H. The P23H mutation results in a mis-folded opsin protein that fails to associate with 11-cis-retinal. The mis-folded P23H protein is retained within cells, where it forms aggregates (Saliba et. al. 2002. JCS 115: 2907-2918; Illing et. a]. 2002. YBC 277: 34150-34160). This aggregation behavior classifies some RIP mutations, including P23H, as protein conformational disorders (PCD).

Previous studies have shown that the native chromophore 11-cis-retinal quantitatively promotes the in vivo folding and stabilization of P23H opsin, as does 9-cis-retinal and a 7-ring locked isomer of 11-cis-retinal (Noorwez et. al. J Biol. Chem. 2003 Apr. 18; 278(16):14442-50). Like the wild-type protein, the rescued mutant P23K protein formed pigment, acquired mature glycosylation and was transported to the cell surface.

### Reagents

Small molecules were procured from National Cancer Institute. Monoclonal anti-rhodopsin 1D4 antibody was purchased from University of British Columbia. β-Ionone was from Sigma and Dodecylmaltopyrannoside (DM) was procured from Anatrace.

### Database Preparation.

The National Cancer Institute/Developmental Therapeutics Program (NCI/DTP) maintains a repository of approximately 220,000 samples (the plated compound set) which are non-proprietary and offered to the extramural research community for the discovery and development of new agents for the treatment of cancer, AIDS, or opportunistic infections afflicting patients with cancer or AIDS (Monga and Sausville 2002). The three-dimensional coordinates for the NCI/DTP plated compound set was obtained in the MDL SD format and converted to the mol2 format by the DOCK utility program SDF2MOL2 (UCSF). Partial atomic charges, solvation energies and van der Waals parameters for the ligands were calculated using SYBDB (Tripos, Inc.) and added to the plated compound set mol2 file.

### Molecular Docking

All docking calculations were performed with the Oct. 15, 2002, development version of DOCK, v5.1.0 (Charifson et al. 1999; Ewing et al. 2001). The general features of DOCK include rigid orienting of ligands to receptor spheres, AMBER energy scoring, GB/SA solvation scoring, contact scoring, internal non-bonded energy scoring, ligand flexibility and both rigid and torsional simplex minimization (Gschwend et al.; Good et al. 1995). Unlike previously distributed versions, this release incorporates automated matching, internal energy (used in flexible docking), scoring function hierarchy and new minimizer termination criteria.

The coordinates for the crystal structure of rhopdopsin, PDB code 1 GZM, was used in the molecular docking calculations. To prepare the site for docking, all water molecules were removed. Protonation of receptor residues was performed with Sybyl (Tripos, St. Louis, Mo.). The structure was explored using sets of spheres to describe potential binding pockets. The number of orientations per molecule was 100. Intermolecular AMBER energy scoring (vdw+columbic), contact scoring and bump filtering were implemented in DOCK5.1.0 (Gschwend et al,). SETOR (Evans 1993) and GRASP (Petrey and Honig 2003) were used to generate molecular graphic images.

### Cell Lines and Culture Conditions

Wild-type (WT) and P23H expressing stable cell lines were generated in a commercially available cloning system, the Flp-In T-Rex System™ (Invitrogen). The stable cells were grown in DMEM high glucose media supplemented with 10% (vlv) fetal bovine serum, antibiotic antimycotic solution, 5 μ/ml blasticidin and hygromycin at 37° C. in presence of 5% CO₂. For all the experiments the cells were allowed to reach confluence and were induced with 1 μg/ml tetracycline after change of media and then compounds were added. The plates were incubated for 48 h after which the cells were harvested.

In Vivo Screening of NCI Compounds that Enhance P23H Opsin Yield

The P23H opsin producing cells were grown in 6-well plates and induced to produce P23H opsin. The DMSO solutions of the NCI compounds were individually added to separate wells at a final concentration of 100 μM. After incubation for 48 h cells were collected and washed with PBS. The cells were then lysed in PBSD by constantly rotating for 1 h at 4° C. The tubes were spun at 36000 rpm for 10 mm and supernatant collected in fresh tubes. Total protein was quantified using a commercially available assay system, the DC protein assay (Biorad). Equal amounts of protein (10 μg) from different samples was loaded on 4-20% SDS polyacrylamide gels and total opsin quantified by western blotting.

### SDS-Page and Western Blotting

Proteins were separated on SDS-PAGE gels and western blotted as described in Noorwez et al. (2004).

### Rhodopsin Purification

For purification of rhodopsin 100 μM MCI 45012 was added to a confluent plate of P23H cells after induction. To another P23H plate β-Ionone was added to a final concentration of 10 μM with an additional dose of 10 μM after 24 h. Rhodopsin purification was essentially as described below for opsin purification with minor modifications. Briefly, the cells were harvested and washed with PBS followed by incubation with 50 μM 11-cis-retinal for 1.5 h at 4° C. The cells were then washed three times with PBS to remove excess retinal. Lysis, column binding and elution from the column were same as in case of opsin purification.

### Example 1

### Use of a Crystal Structure of Rhodopsin

### to Select Potential Modulators

The retinal binding pocket of a trigonal crystal form of bovine rhodopsin, PDB code 1 GZM, was used to identify small molecule modulators by a high throughput molecular docking method. The positions of each retinal atom were used to guide in the definition of the binding pocket selected for molecular docking.

Spheres were positioned at the selected site to allow the molecular docking program, DOCK 5. 1.0 (available from USCF), to match spheres with atoms in potential ligands (small molecules in this ease). During the molecular docking calculation, orientations are sampled to match the largest number of spheres to potential ligand atoms, looking for the low energy structures that bind tightly to the active site of a receptor or enzyme whose active site structure is known.

A scoring grid was calculated to estimate the interaction between potential ligands and the retinal binding pocket target site. The atomic positions and chemical characteristics of residues in close proximity (within 4 angstroms) to the selected site were used to establish a scoring grid to evaluate potential interactions with small molecules. Two types of interactions were scored: van der Waals contact and electrostatic interactions.

DOCK 5.1.0 was used to carry out docking molecular dynamic simulations. The coordinates for approximately 20,000 drug-like compounds (all of which are available through the National Cancer Institute/DTP) were used as the ligand database for molecular docking using the site selected (the retinal binding pocket). These 20,000 compounds were selected from the NCI/DTP collection based on the Lipinski rules for drug likeness. Each small molecule was positioned in the selected site in 100 different orientations, and the best orientations and their scores (contact and electrostatic) were calculated. The scored compounds were ranked and the 20 highest scoring compounds were requested from the NCI/DTP for functional evaluation.

D. Research Design and Methods

D.1 Database Preparation

The National Cancer Institute/Developmental Therapeutics Program (NCI/DTP) maintains a repository of approximately 220,000 samples (the plated compound set) which are non-proprietary and offered to the extramural research community for the discovery and development of new agents for the treatment of cancer, AIDS, or opportunistic infections afflicting patients with cancer or AIDS (Monga and Sausville (2002)). The three-dimensional coordinates for the NCI/DTP plated compound set was obtained in the MDL SD format and converted to the mol2 format by the DOCK utility program SDF2MOL2 ((UCSF). Partial atomic charges, solvation energies and van der Waals parameters for the ligands were calculated using SYBDB (Tripos, Inc.) and added to the plated compound set mol2 file).

D.2 Molecular Docking

All docking calculations were performed with the Oct. 15, 2002, development version of DOCK, v5.1.0 (Charifson et al. 1999; Ewing et al. 2001). The general features of DOCK include rigid orienting of ligands to receptor spheres, AMBER energy scoring, GB/SA solvation scoring, contact scoring, internal non-bonded energy scoring, ligand flexibility and both rigid and torsional simplex minimization (Gschwend et al.; Good et al. 1995). Unlike previously distributed versions, this release incorporates automated matching, internal energy (used in flexible docking), scoring function hierarchy and new minimizer termination criteria.

The coordinates for the crystal structure of rhodopsin, PDB code 1 GZM, were used in the molecular docking calculations. To prepare the site for docking, all water molecules were removed. Protonation of receptor residues was performed with Sybyl (Tripos, St. Louis, Mo.). The structure was explored using sets of spheres to describe potential binding pockets. The number of orientations per molecule was 100. Intermolecular AMBER energy scoring (vdw+columbic), contact scoring and bump filtering were implemented in DOCK 5.1.0 (Gschwend el.). SETOR (Evans 1993) and GRASP (Petrey and Honig 2003) were used to generate molecular graphic images. The approach is generally illustrated in FIG. 1.

### Example 2

### Dot Blot Measurement of Opsin

This example describes measurement of opsin levels in HEK 293 Flp-In™ T-REX™ cell lines (Invitrogen) that are stably transformed with a mutant or wild-type opsin gene or an empty vector. Opsin expression in these cells is inducible with tetracycline. Following induction, cells are lysed in detergent buffer and cellular protein is immobilized on nitrocellulose-containing membranes. Opsin and tubulin (as a loading control) are detected with antibodies and an infrared scanner. This dot procedure can be applied to opsin-containing detergent lysates from other sources, such as mouse eyes.

Cell Growth and Plating (1.5 Days, Starting with Confluent Cells)
1. The following steps must be conducted with aseptic technique in a tissue culture hood.
2. Obtain confluent 10 cm plates of cell lines of Flp-In™ T-REx™ 293 containing the opsin gene and the vector control from plates grown in DMEM containing 10% fetal bovine serum, antibiotic/antimycotic solution, hygromycin and blasticidin and incubated at 37° C. and 8% CO₂.
3. Wash the plates with 10 ml PBS and then treat with 1 ml TrypLE™ Express (Invitrogen 12605-021) for a few minutes. Add 9 ml fresh warm media and resuspend the cells by pipetting up and down five times.
4. Dilute cells 1:2 in fresh warm media.
   a. If only one cell line is to be examined, use a disposable sterile trough and a multipipettor to fill the wells. One plate can be filled with 20 ml of media, so a convenient dilution is 7 ml cells to 14 ml media.
   b. A 24-well plate is useful for dilutions if more than one cell line is used.

Each well of 24-well plate holds 3 ml. For enough cell dilution to fill eight wells on a 96-well plate, a convenient dilution is 0.7 ml to 1.4 ml media.
5. Add 200 μl of diluted cells to desired wells on a sterile round bottom 96-well plate with lid.
   a. Arrange the experiment on the plate to optimize use of the multipipettor. For example, if you are using an 8-channel multipipettor, consider arranging the experiment so that wells in one column of the 96-well plate will all receive the same media.
   b. The plate must include at least six wells of the vector cell line, which are spiked with purified opsin as a standard.
   c. Before dispensing cells into the 96-well plate, pipette up and down several times in the reservoir to be sure they are well suspended.
6. Allow the cells to grow for 36 hours at 37° C., 8% CO₂.
   Opsin Induction (2 Days, Starting with a Confluent 96-Well Plate)
1. Make fresh media containing tetracycline (1 μg/ml) to induce opsin expression. Tetracycline (Invitrogen No. 55-0205) is added from a 1 mg/ml stock in water (1 μl/ml of media). Any test compounds must be paired with an equal volume of the solution in which they are prepared. If required, add 11-cis retinal (R. Crouch via the National Eye Institute) under darkroom lighting at 20 μM from a 20 mM stock in ethanol (1 μl/ml of media). Cells treated with 11-cis retinal or other retinoids that can bind to opsin must be grown in the dark.
2. Using a vacuum aspirator, remove old media. Do not remove media from more than five columns before adding fresh media (either induced or uninduced). This insures that the cells will not dry out.
3. Using a multipettor set to 200 μl, remove media from a loading trough and add it to the wells.
   a. Tilt the open face of the plate toward you so that the sides of the wells are close to level and position the tips at an angle so they rest against the midpoint of the wells. This will minimize disturbance to the cells when the media is added.
   b. Initially add media slowly. As the wells fill up, you may increase the rate of dispensing.
4. Incubate 48 hours at 37° C., 8% CO₂ to induce opsin expression.

### Cell Lysis and Storage (3 Hours)

Remove media and wash cells using warm media base (DMEM) that DOES NOT contain fetal bovine serum (FBS) or antibiotics using same procedure as above under Opsin Induction.
1. Remove media wash and add 200 μl lysis solution (1% w/v dodecyl maltoside (DM) in phosphate-buffered saline (PBS) containing protease inhibitor) to each well:

Stock solutions:

| | |
|---|---|
| protease inhibitor (50×) | 1 tablet Complete Protease |
| | Inhibitor (Roche 11836153001) |
| | dissolved thoroughly in 1.0 ml |
| | H₂O |
| DM (10% w/v) | 5.0 g n-dodecyl-β-D-maltoside |
| | (Anatrace D3110) to 40 ml H₂O, |
| | rock gently to dissolve, adjust to |
| | 50 ml final |
| PBS (10×) | 80 g NaCl, 2 g KCl, 11.5 g |
| | Na₂HPO₄•7H₂O, 2 g KH₂PO₄ |
| | per liter of H₂O |

Lysis solution (22 ml, sufficient for one 96-well plate):

2.2 ml 10% DM

440 μl 50× protease inhibitor

2.2 ml 10×PBS

17.2 ml H₂O
2. Using fresh tips with the multipettor set to 100 μl, thoroughly resuspend the cells in each well.
3. Allow the plate to incubate at room temperature for 2 hours before blotting or storage at −20° C.

### Membrane Transfer (3 Hours)

1. Remove frozen 96-well plates from the −20° C. freezer and remove the lid to prevent drops of condensation from falling back onto the plate. Cover the plate with the lid of a blue tip box to protect it while thawing. Thawing should be complete in about 0.5 h.
2. After the plate has thawed, resuspend the sample in each well by pipetting. If the plate was not frozen and thawed, it may be used without additional pipetting.
3. Place plates into 96-well plate holder and set into CR 312 Jouan centrifuge.
   a. The 96-well plate holders are in the top-left drawer below the centrifuge.
   b. Lower the temperature setting to 4° C., set RPM to 2,000 and spin for 10 min.
4. Cut a 9 cm×12 cm piece of Immobilon-NC membrane (Millipore HAHY00010).
5. Wet membrane by dipping one of the 9 cm edges into COLD PBS (see above for 10×PBS recipe).
   a. Capillary action will pull the PBS up the membrane
   b. As this occurs, progressively lower the membrane onto the PBS surface at a 45° angle without submerging it.
6. Gently shake the wet membrane in 30 ml PBS on the Belly Dancer® (Stovall) at a setting of 3.5 for 10 min to wet any remaining dry spots.
7. Wash the Bio-Dot Microfiltration Apparatus (Biorad 170-6545) and be certain to rinse it with ddH₂O before proceeding.
8. Assemble the apparatus as following:
   a. The vacuum manifold as the base, connected to the tubing and the flow valve.
   b. Place the gasket support plate into the manifold (fits in only one way).
   c. Place the sealing gasket on to, ensuring that all the holds in the gasket line up with the ones on the manifold.
   d. Place the wet membrane on top of the gasket at a 45° angle to lessen the chances of bubbles.
   e. Use a 5 mL pipet to roll out any bubbles
   f. Place the sample template on top of the membrane. Tighten the screws with a diagonal crossing pattern.
   g. Attach a vacuum source to the flow valve
9. Apply the vacuum and tighten the screws, again using the diagonal crossing pattern.
10. Turn the flow valve to atmosphere and add 100 μl of PBS to the center of all the wells.
   a. Adding to center will prevent formation of air bubbles on the bottom of the wells.
   b. Once the wells are empty, raise the dot blot apparatus tube above the level of the drain.
11. Add 1 ml of lysis solution to 10 ml of PBS. Apply 110 μl of this solution to the center of all wells in the dot blot apparatus except those containing lysed vector cells that will be used for the standard curve. To these wells only add 100 μl of PBS.
12. Using fresh yellow tips, remove 20 μl of lysed sample from surface of wells and add to dot blot. To add samples with the multipettor, press the tips against sides of all eight wells, and lower to the bottom corner of the apparatus before dispensing. Pipet up and down to mix the solution.
13. Take the three vials of 10 μl purified rhodopsin (A₅₀₀=0.035, stored in the −80° C. freezer) off ice and add 10 μl of lysis buffer to them. Mix well. Set up an dilution series for the standard curve:
   Purified rhodopsin: 10 μl, 8 μl, 6 μl, 4 μl, 2 μl, 0 μl
   Lysis solution: 0 μl, 2 μl, 4 μl, 6 μl, 8 μl, 10 μl
14. Lower dot blot apparatus tub and place so that tip is just off the edge of the counter. The outlet should be positioned so that it takes approximately 30 min to drain.
15. After samples have drained, add 400 μl of PBS to all wells, hang the drainage tube all the way over the edge of the counter. It should take approximately 1 hour to drain.
16. After drained, unscrew dot blot top and place membrane into a suitable container (for example, the lid from a Bio-Rad Centurion gel package) and add blocking buffer (15 ml of LI-COR Odyssey® blocking buffer to 15 ml of PBS).

a. Place membrane on belly dancer at setting 3.5 for minimum of 1 hour.

b. Put on cold room shaker if you want to leave it overnight or longer.

### Immunodetection (3 Hours)

1. All of the following incubations are at room temperature.
2. Remove blocking buffer and add 30 ml of PBST (1 ml of Tween-20 per liter of PBS).
3. Add 15 μl of anti-tubulin antibody and 30 μl of anti-opsin antibody.
   a. Rabbit polyclonal to β-tubulin is the anti-tubulin antibody (Abcam ab6046-200).
   b. Mouse Rho ID4 purified monoclonal antibody (University of British Columbia) is the anti-opsin antibody.
   c. Place on belly dancer for 1 hour.
   d. After 1 hour, remove antibodies, and wash 3 times with 30 ml PBST for 5 minutes each.
4. Add 30 ml PBST, then secondary antibodies.
   a. Add 30 μl of Alexa Fluor® 680 goat anti-rabbit IgG (H+L) (Invitrogen A21109).
   b. Add 30 μl IR Dye® 800 Conjugated Affinity Purified anti-mouse IgG (H+L) (Goat). (Rockland 610-132-121).
   c. Place on belly dancer for 1 hour.
   d. Wash 3 times with 30 ml PBST for 5 minutes each.
      Scanning and Data Analysis (0.5 h with the Excel Template)
1. Scan the blot on the LI-COR® Odyssey Infrared Imaging System.
   a. The default resolution and image quality is adequate for quantitation.
   b. If saturation is observed in the 700 nm or 800 nm channel decrease the scan intensity and restart.
   c. Scan a 9×12 grid so that the grid function (see below) will be correctly sized to the wells.
2. Analyze the data (for example, using Excel or other statistical analysis software).
   a. Use the 96-well grid function in, for example, the ODYSSEY® Infrared Imaging System (LI-COR® Biosciences, Lincoln, Nebr.) to superimpose a grid on the image for each well of the blot.
   b. Use the Grid Sheet function to transfer 700 nm and 800 nm integrated pixel intensities to separate sheets of a Microsoft Excel worksheet.
   c. In a third sheet of the worksheet, divide each 800 nm value by the corresponding 700 nm value for all wells in the plate, including the standard curve.
   d. On this sheet, average the standard curve data for each rhodopsin load volume and create a table containing the load volumes, the 800 nm/700 nm values, and the standard deviation of the values. Plot these data and fit the plot with a second-order polynomial (y=ax²+bx+c).
   e. In a fourth sheet, use the coefficients a, b, and c from fitting the standard curve to obtain the positive solution of the quadratic formula for each value y in sheet three (800 nm/700 nm). This operation yields the opsin level in each sample, normalized to total tubulin and corrected for non-linear behavior in blotting, immunodetection or imaging.
   f. Average the replicates and normalize to the appropriate controls to obtain the relative change in opsin levels that occur as a result of treatment. In Excel, apply a two-tailed Student's t-test for samples with unequal variance to determine the significance of the change (the P value should be less than 0.05 for statistical significance).

### Example 3

### Effect of Compounds on P23H Rhodopsin Yield

The ability of candidate compounds to affect the yield of P23H rhodopsin is believed to be indicative of the ability of the compound to stabilize the mutant opsin. See generally Noorwez et al. (2004) and U.S. Patent Publication No. 2004-0242704, both of which are incorporated herein by reference.

Cell Lines and Culture Conditions.

Stable cell lines expressing the P23H opsin were generated using Flp-In T-Rex system (Invitrogen) in HEK293 cell line. To create plasmids for constructing stable cell lines, an EcoRI-NotI fragment from the wild-type or P23H mutant synthetoc bovine opsin gene in pMT4 (see Kaushal et al., Biochemistry, Vol. 33, pp. 6121-6128 (1994) was combined with the large EcoRI-NotI fragment of pcDNA5/FRT/TO (Invitrogen). The resulting plasmids contain opsin under the control of a tetracycline-inducible human cytomegalovirus promoter and a flippase recognition sequence (FRT) for site sepecific recombination at the unique chromosomal FRT site of HEK293 Flp-In T-Rex. The opsin sequence in these plasmids was verified by PCR cycle sequencing. To construct stable cell lines, HEK293 Flp-In T-Rex cells were co-transfected with the flippase vector pOG44 (from Invitrogen) and the pcDNA5/FRT/TO vector or its derivatives containing the opsin gene. Stable recombinants were obtained by selecting for cells expressing resistance to hygromycin due to plasmid recombination at the FRT site. After the initial selection, the stable cell lines were routinely grown in the same media as HEK293 Flp-In T-rex, except that hygromycin was substituted for zeocin.

The stable cells were grown in DMEM high glucose media supplemented with 10% (v/v) fetal bovine serum, antibiotic/antimycotic solution, 5 μg/ml blasticidin and hygromycin at 37° C. in presence of 5% CO₂. For all the experiments the cells were allowed to reach confluence and were induced with 1 μg/mi tetracycline after change of media and then compounds were added. The plates were incubated for 48 h after which the cells were harvested.

Opsin Purification, Regeneration and Retinal Competition.

WT opsin producing cells were grown in 10 cm culture plates and induced for opsin production. After 48 hour of induction the cells were harvested and washed with PBS (10 mM sodium phosphate, 130 mM NaCl, pH 7.2). The cells were lysed in PBSD (PBS containing 1.0% DM and 1× protease inhibitor cocktail) for 1 hour and the lysate was added to 1D4 coupled sepharose beads and incubated for 1 hour at 4° C. The beads were washed 5 times in PBSD and twice with 10 mM sodium phosphate buffer containing 0.5% DM, pH 6.0. The bound WT opsin was eluted with a competing peptide representing the last 18 amino acids of rhodopsin in the latter buffer for 1 hour. The purified opsin was immediately used for regeneration with 11-cis retinal and for competition studies with the selected NCI compounds and β-ionone.

All the opsin regeneration and competition assays were performed under dim red light in a Cary 50 spectrophotometer (Varian) equipped with temperature control. 25 μM purified WT opsin was mixed with 50 μM 11-cis retinal and scanned every two minutes in the range of 250-650 nm until no more rhodopsin was regenerated. Similarly opsin was mixed with compound SN10011 (2 or 5 mM) and allowed to sit for 10 minutes on ice. Then retinal was mixed and spectra taken every two minutes. β-Ionone was used at 5 and 50 μM concentrations. The reactions were conducted in 100 μl total volume where the compounds were provided in 2 μl solution to 98 μl opsin solution. The temperature was maintained at 20° C.

Effect of Compounds.

The P23H opsin producing cells were grown in 6-well plates and induced to produce P23H opsin. The DMSO solutions of the test compounds were individually added to separate wells at a final concentration of 100 μM. After incubation for 48 h cells were collected and washed with PBS. The cells were then lysed in PBSD by constantly rotating for 1 hour at 4° C. The tubes were spun at 36,000 rpm for 10 mm and supernatant collected in fresh tubes. Total protein was quantified using DC protein assay (Biorad). Equal amounts of protein (10 μg) from different samples was loaded on 4-20% SDS polyacrylamide gels and total opsin quantified by western blotting. Proteins were separated on SDS-PAGE gels and western blotted as described in Noorwez et at (2004).

Rhodopsin Purification.

The effect of the compounds on P23H rhodopsin yield was assessed by purifying and spectrophotormetrically determining the yield. After 48 hours of induction and treatment with compounds the cells were harvested and rhodopsin purified essentially as described in Noorwez et al. (2004). For purification of rhodopsin 100 μM NCI-45012 was added to a confluent plate of P23H cells after induction. To another P23H plate β-Ionone was added to a final concentration of 10 μM with an additional dose of 10 μM after 24 h. Rhodopsin purification was essentially as described earlier for opsin purification with minor modifications. Briefly, the cells were harvested and washed with PBS followed by incubation with 50 μM 11-cis-retinal for 1.5 hours at 4° C., The cells were then washed three times with PBS to remove excess retinal. Lysis, column binding and elution from the column were the same as for opsin purification.

Results

Compounds identified by computational modeling (see Example 1) were screened as described above. The effect of compounds on the yield of mutant rhodopsin was determined by spectrophotometry. Exemplary absorbance spectra showing the effect of Compound 1 on the yield of mutant P23H rhodopsin are depicted in FIG. 2.

Compounds showing at least a 15% increase in the yield of P23H rhodopsin (compared to 10 control) at a compound concentration of 100 μM included compounds 1 to 6 (shown below):

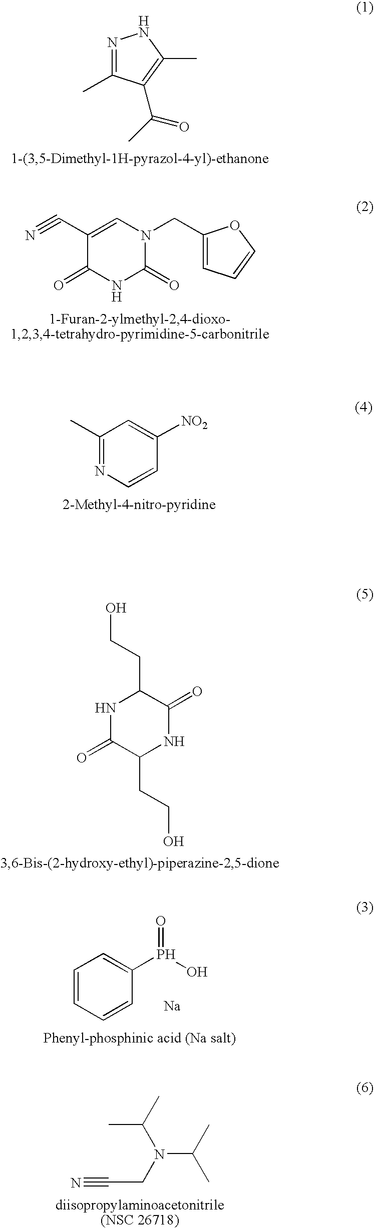

TABLE 1

| Compound No. | % Increase in P23H Yield |
|---|---|
| 1 | 31 |
| 2 | 43 |
| 3 | 32 |
| 4 | 24 |
| 5 | 15 |
| 6 | 28 |

TABLE 2

| | % Increase in |
|---|---|
| Compound (NSC No.) | P23H Yield |
| 26718 (Compound 6) | 25 ± 3 |
| 27009 (3,4-Methylenedioxybenzonitrile) | 20 ± 5 |
| 45012 (Compound 1) | 30 ± 5 |
| 47520 (Compound 2) | 40 ± 7 |
| 49193 (Diethyl-(2-mercaptoethyl)-amine) | 15 ± 6 |
| 66688 (6-imino-1-methyl-1,6-dihydro-3- | 40 ± 9 |
| pyridinecarboxamine) | |
| 114498 (1H-1,2,3-benzotriazol-1-amine) | 30 ± 6 |
| 121968 (4-Salicylideneamino-1,2,4-triazol) | 29 ± 5 |
| 163936 (Compound 3) | 40 ± 3 |
| 170691 (Compound 4) | 25 ± 8 |
| 227405 (Compound 5) | 30 ± 10 |

### Example 4

### Effect of β-Ionone on Opsin Regeneration

The structure of β-ionone is as follows:

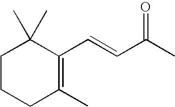

As shown in FIG. 1, β-ionone inhibits opsin regeneration and rescues mutant opsin in vitro. β-ionone (10 μM) was added to cells producing P23H mutant opsin and incubated for 48 hours. When the mutant opsin was synthesized in continuous presence of β-ionone, a 2.5-fold increase in pigment was observed (FIG. 1b) compared to when no β-ionone was present. This increase in pigment was then compared with that generated by 11-cis-retinal, which has been shown to pharmacologically rescue mutant opsin (Noorwez et al. (2004)), by producing mutant opsin in the presence of 11-cis-retinal. A 5-fold increase in pigment was obtained with 11-cis-retinal. Without wishing to be bound by theory, this difference in the level of rescue might be due to the truncation of the side chain in the case of β-ionone and the capability of forming a covalent bond, which is a much stronger anchor.

To determine whether a 500 nm absorbing pigment is formed upon addition of β-ionone, purified wt (wild-type) opsin was mixed with β-ionone, incubated for 15 minutes, and scanned for pigment formation (FIG. 1c). β-ionone does not form a light absorbing pigment with opsin. To determine if any pigment is generated with β-ionone in HEK293 cells the mutant opsin was expressed in these cells and β-ionone was added at the time of opsin induction. The cells were washed after 48 h and rhodopsin purified without treating the cells with 11-cis-retinal under conditions that yield only folded rhodopsin (Noorwez et al. (2004)). No pigment was detected, signifying that β-ionone was not processed by the HEK cells into retinoids that could produce pigment with opsin.

To determine whether this rescue by β-ionone also affects the partitioning of more P23H molecules towards the folded state, the effect of β-ionone on the total yield of opsin versus that of properly folded P23H rhodopsin was determined by quantitative western blotting (FIG. 1d). The addition of β-ionone increased the yield of total opsin. Separate comparison of the pool of properly folded native-like P23H rhodopsin showed that the increase was greater. Thus, a higher fraction of P23H molecules attain a stable conformation. In sum, the increase in yield of total opsin in the presence of 11-cis-retinal was 2.4 fold and the yield of folded rhodopsin was 2.6 fold, indicating the efficiency of β-ionone in stabilizing the mutant opsin. This might also explain the difference in the degree of rescue obtained with these two pharmacological chaperones. The data are shown in FIG. 1.

Here we have demonstrated that smaller molecules, e.g., β-ionone, that non-covalently bind to the chromophore binding site of opsin, thus inhibiting binding of retinal to the site, are capable of functioning as pharmacological chaperones. Similar results have been found for cis-1,3-dimethylcyclohexane. It is important to note that these compounds, although pharmacological chaperones, are non-retinoids. In vivo studies reveal that these compounds increase the cellular yields of folded P23H rhodopsin. Although the molecular docking strategy is a powerful tool for the discovery of inhibitors, the present invention demonstrates a novel utility for the power of high-throughput in silico screening combined with functional testing in identifying novel pharmacological chaperones for ocular protein conformation disorders. Such functional testing is recited in the screening methods of the invention. Thus, in silico methods have proved useful in identifying types of non-retinoid molecules that might prove useful in correcting mis-folding in opsins. Once identified, these compounds exhibited selective binding properties in their interaction with opsin and the screening methods of the invention take advantage of these properties to find other compounds binding through a similar mechanism as a means of identifying potential therapeutic agents.

In these experiments, rhodopsin was purified under conditions that selectively yield properly folded, 11-cis-retinal bound opsin. These data also show that 11-cis-retinal was about 2-fold more effective than β-ionone in increasing the cellular yields of P23H rhodopsin (FIG. 1). This difference probably reflects the inability of β-ionone to form stable Schiff base linkage with lysine 296 in the protein.

Since HEK293 cells are known to possess a retinoid processing machinery, opsin was purified from β-ionone treated cells and spectroscopically analyzed for formation of pigment to determine whether β-ionone is processed by the cells to form any pigment. No pigment was observed when opsin was purified from β-ionone treated cells (FIG. 1e—solid line). Pigment was observed only after treating the cells with 11-cis-retinal (FIG. 1e—dashed line). To further test the hypothesis that compounds that non-covalently bind to the chromophore binding site lead to pharmacological rescue of the mutant protein, rhodopsin was purified from P23H opsin expressing cells that were treated with cis-1,3-dimethylcyclohexane, a much weaker inhibitor of opsin regeneration with 11-cis-retinal. Presence of cis-1,3-dimethylcyclohexane led to a 15% increase in the yield of P23H rhodopsin (FIG. 1f). The lower yield of rhodopsin in the presence of this compound is consistent with its weaker inhibitory capacity.

Collectively, these results indicate that small compounds that fit into the retinal binding pocket of opsin and/or compete with 11-cis-retinal in vitro are useful as pharmacological chaperones.

### Example 4

### Effect of SN10011 on Opsin Regeneration

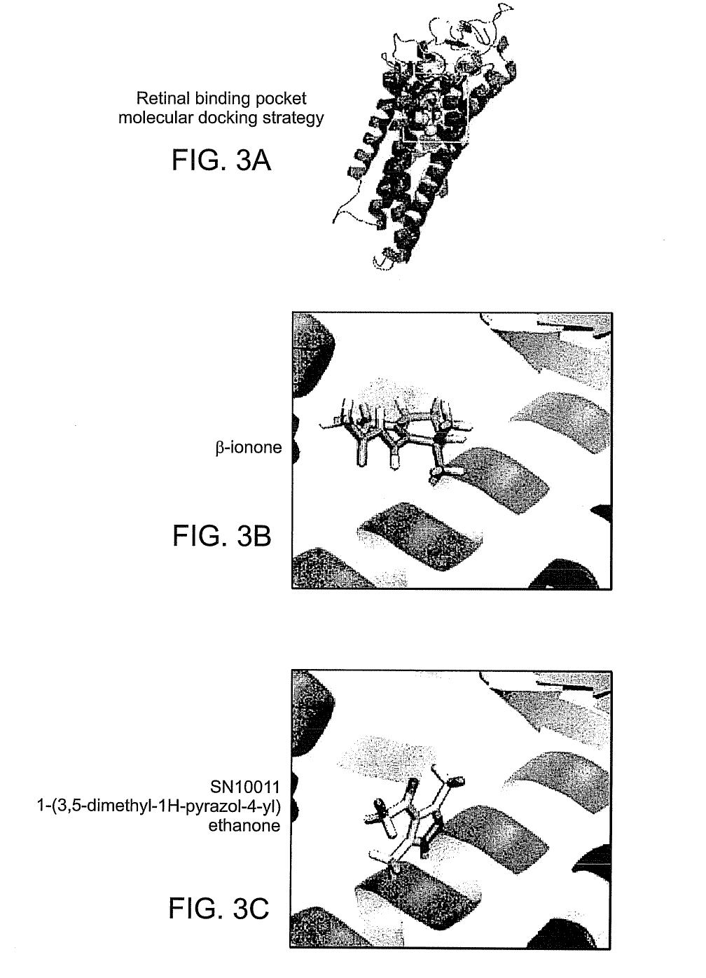

To identify non-retinoid compounds that could be useful therapeutic agents, we performed molecular docking using a large chemical library of drug-like small molecules in the National Cancer Institute Developmental Therapeutics Program. DOCK5.1 (UCSF) was used to position each one of 20,000 drug-like compounds into the selected site. Each compound was positioned in 100 different orientations, and the best scoring orientations were obtained. Unlike previous molecular docking strategies, each docked compound was selected based on chemical criteria; the Lipinski rules for drug likeness, which are described, for example, by Lipinski et al., Adv Druq Deliv Rev. 2001 Mar. 1; 46(1-3):3-26. Therefore, this strategy eliminates compounds that are less likely to be developed into therapeutic agents. FIG. 3C shows the fifth highest scoring compound, 1-(3,5-dimethyl-1H-pyrazol-4-yl)ethanone, SN10011, in the orientation posed by DOCK5.1 (UCSF) at or in the retinal binding pocket based on the crystal structure of rhodopsin. Compound SN10011 inhibits opsin regeneration and rescues mis-folded opsin.

We tested the top scoring compounds (the highest 0.05% energy scores for their effect as inhibitors of opsin regeneration. One compound, SN10011 showed a significant effect on inhibition of pigment formation with 11-cis-retinal. The effect of SN10011 was studied by addition of 2 and 5 mM SN10011 to the opsin solution followed by addition of 11-cis-retinal (FIG. 2a). Presence of this compound increased the t_{1/2} from 5 minutes to 8 minutes (2 mM) and 12 min (5 mM), respectively. This demonstrates a dose dependence of regeneration inhibition. The extent of inhibition was much lower than that obtained with β-ionone and the concentrations of this compounded needed to reach the observable inhibition levels were also much higher than that of β-ionone. To test whether this compound associates with WT opsin to form pigment it was added to opsin solution in vitro. No pigment was formed by SN10011 with WT opsin (FIG. 2b) and by itself the compound does not show any absorption in the visible spectrum (FIG. 2c).

To test the principle that a regeneration inhibitor that occupies the retinal binding site of opsin should rescue the misfolded protein, compound SN10011 was added to cells producing P23H mutant opsin. A 30% increase in the yield of folded P23H rhodopsin was achieved in presence of SNIOO11 (FIG. 2d). The increase in yield is much smaller than that achieved with β-ionone which corresponds well with it being a weaker inhibitor of regeneration than β-ionone.

We have utilized a high-throughput computer-based molecular docking approach that made use of the coordinates of the retinal binding site coupled with functional studies in vitro and in vivo to identify 1-(3,5-dimethyl-1H-pyrazol-4-yl)ethanone (SN10011), a drug-like small molecule, that inhibits the binding of 11-cis-retinal to opsin in vitro, suggesting that the identified molecules occupy the retinal binding pocket.

These results suggest that the rescue of P23H opsin by this compound emanates from it being an opsin regeneration inhibitor whereby it stabilizes the mutant opsin (although with low efficiency).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### REFERENCES

Abagyan et al., Curr. Opin. in Chemical Biology 5(4): 375-82 (2001).
Bernier et al., Trends Endocrin. Metab 15, 222-228 (2004).
Berson, E. L. Int. Opthalmol. Clin. 40, 93-111 (2000).
Boehm et al., J. Med. Chem. 43(14): 2664-74 (2000).
Charifson et al., Journal of Medicinal Chemistry 42(25): 5100-9 (1999).
Collins et al., Annu. Rev. Pharmacol. Toxicol. 39, 399-430 (1999).
Doman et al., J. Med. Chem. 45(11): 2213-21 (2002).
Enyedy et al., J. Med. Chem. 44(9): 1349-55 (2001).
Enyedy et al., J. Med. Chem. 44(25): 4313-24 (2001).
Evans, S. V., J. Mol. Graphics 11(2): 134-8, 127-8 (1993).
Ewing et al., Journal of Computer-Aided Molecular Design 15(5): 411-28 (2001).
Freymann et al., Chemistry & Biology 7(12): 957-68 (2000).
Good et al., Journal of Computer-Aided Molecular Design 9(I): 1-12 (1995).
Gruneberg et al., J. Med. Chem. 45(17): 3588-602 (2002).
Gschwend et al., Journal of Molecular Recognition 9(2): 175-86 (1996).
Honma et al., J. Med. Chem. 44(26): 4615-27 (2001).
Honma et al., J. Med. Chem. 44(26): 4628-40 (2001).
Iwata et al., J. Med. Chem. 44(11): 1718-28 (2001).
Krebs et al., Trends Biochem. Sci. 29, 648-655 (2004).
Kuksa et al., J. Biol. Chem. 277, 42315-42324 (2002).
Li et al., Proc. Natl. Acad. Sci (USA) 95, 11933-11938 (1998).
Liebeschuetz et al., J. Med. Chem. 45(6): 1221-32 (2001).
Monga et al., Leukemia: Official Journal of the Leukemia Society of America, Leukemia Research Fund, U.K 16(4): 520-6 (2002).
Noorwez et al., J. Biol. Chem. 278, 14442-14450 (2003).
Noorwez et al., J. Biol. Chem. 279, 16278-16284 (2004).
Paiva et al., Biochimica Biophysica Acta 1545(1-2): 67-77 (2001).
Pang et al., FEBS Letters 502(3): 93-97 (2001).
Petrey et al., Meth. Enzym. 374:492-509 (2003)
Saliba et al., J. Cell Sci., 115, 2907-2918 (2002).
Schapira et al., PNAS 97(3): 1008-13 (2000).
Shoichet et al., Curr. Opin. in Chem. Biol. 6(4): 439-46 (2002).
Teelmann et al., Pharmacol. Ther. 40, 29-43 (1989).

1. A method of correcting the conformation of a mis-folded opsin protein, comprising contacting a mis-folded opsin protein with an opsin-binding agent that reversibly binds non-covalently to said mis-folded opsin protein, thereby correcting the conformation of said mis-folded opsin protein.

2. The method of claim 1, wherein said opsin binding agent is selective for opsin.

3. The method of claim 1, wherein said opsin-binding agent competes with a retinoid for binding to said opsin.

4. The method of claim 1, wherein said opsin-binding agent binds in the retinal binding pocket of said opsin.

5. The method of claim 1, wherein said opsin-binding agent binds to said opsin protein so as to inhibit covalent binding of 11-cis-retinal to said opsin protein when said 11-cis-retinal is contacted with said opsin protein when said non-retinoid opsin-binding agent is present.

6. The method of claim 1, wherein said opsin-binding agent is a non-retinoid.

7-10. (canceled)

11. The method of claim 1, wherein said mis-folded opsin protein comprises a mutation in its amino acid sequence selected from the group consisting of T17M, P347S and P23H.

12-13. (canceled)

14. The method of claim 1, wherein the opsin-binding agent is selected from the group consisting of 1-(3,5-dimethyl-1H-pyrazol-4-yl)-ethanone, 1-furan-2-ylmethyl-2,4-dioxo-1,2,3,4-tetrahydro-pyrimidine-5-carbonitrile, phenyl-phosphinic acid, 2-methyl-4-nitro-pyridine, 3,6-bis-(2-hydroxyethy)-piperazine-2,5-dione, diisopropylaminoacetonitrile, 3,4-methylenedioxybenzonitrile, diethyl(2-mercaptoethyl)amine, 6-imino-1-methyl-1,6-dihydro-3-pyridinecarboxamide, 1H-1,2,3-benzotriazol-1-amine, 4-salicylideneamino-1,2,4-triazole, β-ionone, cis-1,3-dimethylcyclohexane, and a pharmaceutically acceptable salt, hydrate, or solvate thereof.

15. A method of rescuing photoreceptor function in a mammalian eye containing a mis-folded opsin protein, comprising contacting said mis-folded opsin protein with an opsin-binding agent that reversibly binds non-covalently to said mis-folded opsin protein, thereby rescuing photoreceptor function in said mammalian eye.

16-27. (canceled)

28. The method of claim 12, wherein the non-retinoid opsin-binding agent is selected from the group consisting of 1-(3,5-dimethyl-1H-pyrazol-4-yl)-ethanone, 1-furan-2-ylmethyl-2,4-dioxo-1,2,3,4-tetrahydro-pyrimidine-5-carbonitrile, phenyl-phosphinic acid, 2-methyl-4-nitro-pyridine, 3,6-bis-(2-hydroxyethy)-piperazine-2,5-dione, diisopropylaminoacetonitrile, 3,4-methylenedioxybenzonitrile, diethyl(2-mercaptoethyl)amine, 6-imino-1-methyl-1,6-dihydro-3-pyridinecarboxamide, 1H-1,2,3-benzotriazol-1-amine, 4-salicylideneamino-1,2,4-triazole, β-ionone, cis-1,3-dimethylcyclohexane, and a pharmaceutically acceptable salt thereof.

29. A method of stabilizing a mutant opsin protein in a wild-type protein conformation, comprising contacting said mutant opsin protein with an opsin-binding agent that reversibly binds non-covalently to said mutant opsin protein, thereby stabilizing said mutant opsin protein in a wild-type protein conformation.

30-41. (canceled)

42. The method of claim 41, wherein the non-retinoid opsin-binding agent is selected from the group consisting of 1-(3,5-dimethyl-1H-pyrazol-4-yl)-ethanone, 1-furan-2-ylmethyl-2,4-dioxo-1,2,3,4-tetrahydro-pyrimidine-5-carbonitrile, phenyl-phosphinic acid, 2-methyl-4-nitro-pyridine, 3,6-bis-(2-hydroxyethy)-piperazine-2,5-dione, diisopropylaminoacetonitrile, 3,4-methylenedioxybenzonitrile, diethyl(2-mercaptoethyl)amine, 6-imino-1-methyl-1,6-dihydro-3-pyridinecarboxamide, 1H-1,2,3-benzotriazol-1-amine, 4-salicylideneamino-1,2,4-triazole, β-ionone, cis-1,3-dimethylcyclohexane, and a pharmaceutically acceptable salt thereof.

43. A method of ameliorating an ocular protein conformation disease in a subject, comprising administering to the subject an effective amount of an opsin-binding agent that reversibly binds non-covalently to said mutant opsin protein, thereby ameliorating the ocular protein conformation disease.

44-48. (canceled)

49. The method of claim 43, wherein said mammal has or has a propensity to develop an ocular protein conformation disease selected from the group consisting of the wet or dry form of age-related macular degeneration, retinitis pigmentosa, retinal or macular dystrophy, Stargardt's disease, Sorsby's dystrophy, autosomal dominant drusen, Best's dystrophy, peripherin mutation associated with macular dystrophy, a dominant form of Stargart's disease, North Carolina macular dystrophy, light toxicity, and retinitis pigmentosa.

50-55. (canceled)

56. The method of claim 43, wherein the opsin-binding agent is selected from the group consisting of 1-(3,5-dimethyl-1H-pyrazol-4-yl)-ethanone, 1-furan-2-ylmethyl-2,4-dioxo-1,2,3,4-tetrahydro-pyrimidine-5-carbonitrile, phenyl-phosphinic acid, 2-methyl-4-nitro-pyridine, 3,6-bis-(2-hydroxyethy)-piperazine-2,5-dione, diisopropylaminoacetonitrile, 3,4-methylenedioxybenzonitrile, diethyl(2-mercaptoethyl)amine, 6-imino-1-methyl-1,6-dihydro-3-pyridinecarboxamide, 1H-1,2,3-benzotriazol-1-amine, 4-salicylideneamino-1,2,4-triazole, β-ionone, cis-1,3-dimethylcyclohexane, and a pharmaceutically acceptable salt thereof.

57. An opthalmologic composition comprising an effective amount of an opsin-binding agent in a pharmaceutically acceptable carrier, wherein said agent reversibly binds non-covalently to opsin protein to prevent retinoid binding in the retinal binding pocket of said opsin.

58-62. (canceled)

63. The composition of claim 57, wherein the opsin-binding compound is selected from 1-(3,5-dimethyl-1H-pyrazol-4-yl)-ethanone, 1-furan-2-ylmethyl-2,4-dioxo-1,2,3,4-tetrahydro-pyrimidine-5-carbonitrile, phenyl-phosphinic acid, 2-methyl-4-nitro-pyridine, 3,6-bis-(2-hydroxyethy)-piperazine-2,5-dione, diisopropylaminoacetonitrile, 3,4-methylenedioxybenzonitrile, diethyl(2-mercaptoethyl)amine, 6-imino-1-methyl-1,6-dihydro-3-pyridinecarboxamide, 1H-1,2,3-benzotriazol-1-amine, 4-salicylideneamino-1,2,4-triazole, β-ionone, cis-1,3-dimethylcyclohexane, and a pharmaceutically acceptable salt thereof.

64. An oral dosage form comprising a non-retinoid agent of claim 57.

65-74. (canceled)

75. A method for treating or preventing an ocular protein conformation disease in a subject, comprising administering to a subject having or at risk of developing an ocular protein conformation disease a therapeutically effective amount of an opsin-binding agent selected from the group consisting 1-(3,5-dimethyl-1H-pyrazol-4-yl)-ethanone, 1-furan-2-ylmethyl-2,4-dioxo-1,2,3,4-tetrahydro-pyrimidine-5-carbonitrile, phenyl-phosphinic acid, 2-methyl-4-nitro-pyridine, 3,6-bis-(2-hydroxyethy)-piperazine-2,5-dione, diisopropylaminoacetonitrile, 3,4-methylenedioxybenzonitrile, diethyl(2-mercaptoethyl)amine, 6-imino-1-methyl-1,6-dihydro-3-pyridinecarboxamide, 1H-1,2,3-benzotriazol-1-amine, 4-salicylideneamino-1,2,4-triazole, β-ionone, cis-1,3-dimethylcyclohexane, and a pharmaceutically acceptable salt thereof.

76. The method of claim 75, wherein the ocular protein conformation disorder is selected from the group consisting of wet or dry form of macular degeneration, retinitis pigmentosa, a retinal or macular dystrophy, Stargardt's disease, Sorsby's dystrophy, autosomal dominant drusen, Best's dystrophy, peripherin mutation associate with macular dystrophy, dominant form of Stargart's disease, North Carolina macular dystrophy, light toxicity, and retinitis pigmentosa.

77-80. (canceled)

81. The method of claim 75, wherein the non-retinoid opsin-binding agent is selected from the group consisting of 1-(3,5-dimethyl-1H-pyrazol-4-yl)-ethanone, 1-furan-2-ylmethyl-2,4-dioxo-1,2,3,4-tetrahydro-pyrimidine-5-carbonitrile, phenyl-phosphinic acid, 2-methyl-4-nitro-pyridine, 3,6-bis-(2-hydroxyethy)-piperazine-2,5-dione, diisopropylaminoacetonitrile, 3,4-methylenedioxybenzonitrile, diethyl(2-mercaptoethyl)amine, 6-imino-1-methyl-1,6-dihydro-3-pyridinecarboxamide, 1H-1,2,3-benzotriazol-1-amine, 4-salicylideneamino-1,2,4-triazole, β-ionone, cis-1,3-dimethylcyclohexane, and a pharmaceutically acceptable salt thereof.

82-91. (canceled)

92. A method of increasing the amount of biochemically functional opsin protein in a photoreceptor cell, comprising contacting a photoreceptor cell with an effective amount of an opsin-binding agent that reversibly binds non-covalently to an opsin protein in said cell, thereby increasing the level of biochemically functional conformation of opsin protein.

93-103. (canceled)

104. The method of claim 92, wherein the non-retinoid opsin-binding agent is selected from the group consisting of 1-(3,5-dimethyl-1H-pyrazol-4-yl)-ethanone, 1-furan-2-ylmethyl-2,4-dioxo-1,2,3,4-tetrahydro-pyrimidine-5-carbonitrile, phenyl-phosphinic acid, 2-methyl-4-nitro-pyridine, 3,6-bis-(2-hydroxyethy)-piperazine-2,5-dione, diisopropylaminoacetonitrile, 3,4-methylenedioxybenzonitrile, diethyl(2-mercaptoethyl)amine, 6-imino-1-methyl-1,6-dihydro-3-pyridinecarboxamide, 1H-1,2,3-benzotriazol-1-amine, 4-salicylideneamino-1,2,4-triazole, β-ionone, cis-1,3-dimethylcyclohexane, and a pharmaceutically acceptable salt thereof.

105. A method of correcting the conformation of a mis-folded opsin protein, comprising contacting a mis-folded opsin protein with a retinoid opsin-binding agent that binds to said mis-folded opsin protein in the retinal binding pocket of said opsin, thereby correcting the conformation of said mis-folded opsin protein.
